(19) United States
(12) Patent Application Publication
(10) Pub. No.: US 2010/0094320 A1
(43) Pub. Date: Apr. 15, 2010

(54) Atherectomy and Thrombectomy Devices, Methods for Making, and Procedures for Using

(75) Inventors: Vacit Arat, La Canada Flintridge, CA (US); Richard T. Cohen, Woodland Hills, CA (US); Ming Ting Wu, Encino, CA (US); Pavel B. Lembrikov, Santa Monica, CA (US); Adam L. Cohen, Los Angeles, CA (US)

Correspondence Address: MICROFABRICA INC.;ATT: DENNIS R. SMALLEY, 7911 HASKELL AVENUE, VAN NUYS, CA, 91406, US

(73) Assignee: Microfabrica Inc.

(21) Appl. No.: 12/614,006

(22) Filed: Nov. 6, 2009

Related U.S. Application Data
(63) Continuation of application No. 12/203,126, filed on Sep. 2, 2008, now abandoned.
Application No. 12/203,126 is a continuation-in-part of application No. 12/198,073, filed on Aug. 25, 2008.
Application No. 12/198,073 is a continuation-in-part of application No. 12/179,573, filed on Jul. 24, 2008.
Application No. 12/198,073 is a continuation-in-part of application No. 11/734,273, filed on Apr. 11, 2007.
Application No. 11/734,273 is a continuation-in-part of application No. 12/179,573, filed on Jul. 24, 2008.
Application No. 12/179,573 is a continuation-in-part of application No. 12/134,188, filed on Jun. 5, 2008.
Application No. 12/134,188 is a continuation-in-part of application No. 11/625,807, filed on Jan. 22, 2007.
Application No. 11/625,807 is a continuation-in-part of application No. 12/144,618, filed on Jun. 23, 2008.
Application No. 12/144,618 is a continuation-in-part of application No. 12/179,295, filed on Jul. 24, 2008.
Application No. 12/179,295 is a continuation-in-part of application No. 11/598,968, filed on Nov. 14, 2006.
Application No. 11/598,968 is a continuation-in-part of application No. 11/582,049, filed on Oct. 16, 2006.
Application No. 11/582,049 is a continuation-in-part of application No. 11/444,999, filed on May 31, 2006.
Application No. 11/444,999 is a continuation-in-part of application No. 10/697,598, filed on Oct. 29, 2003, now abandoned.
Application No. 10/697,598 is a continuation-in-part of application No. 11/591,911, filed on Nov. 1, 2006.
Application No. 11/591,911 is a continuation-in-part of application No. 10/697,598, filed on Oct. 29, 2003, now abandoned.
Application No. 12/179,295 is a continuation-in-part of application No. 12/169,528, filed on Jul. 8, 2008.
Application No. 12/169,528 is a continuation-in-part of application No. 12/144,618, filed on Jun. 23, 2008.
Application No. 12/203,126 is a continuation-in-part of application No. 11/734,273, filed on Apr. 11, 2007.
Application No. 11/734,273 is a continuation-in-part of application No. 12/144,618, filed on Jun. 23, 2008.
(60) Provisional application No. 60/969,155, filed on Aug. 30, 2007.
(60) Provisional application No. 61/034,916, filed on Mar. 7, 2008.
(60) Provisional application No. 60/968,030, filed on Aug. 24, 2007.
(60) Provisional application No. 60/968,006, filed on Aug. 24, 2007.
(60) Provisional application No. 60/942,200, filed on Jun. 5, 2007.
(60) Provisional application No. 60/943,310, filed on Jun. 12, 2007.
(60) Provisional application No. 60/949,850, filed on Jul. 14, 2007.
(60) Provisional application No. 60/951,711, filed on Jul. 24, 2007.
(60) Provisional application No. 60/968,042, filed on Aug. 24, 2007.
(60) Provisional application No. 61/018,283, filed on Dec. 31, 2007.
(60) Provisional application No. 60/945,570, filed on Jun. 21, 2007.
(60) Provisional application No. 60/951,707, filed on Jul. 24, 2007.
(60) Provisional application No. 60/968,043, filed on Aug. 24, 2007.
(60) Provisional application No. 61/018,303, filed on Dec. 31, 2007.
(60) Provisional application No. 60/761,401, filed on Jan. 20, 2006.
(60) Provisional application No. 60/736,961, filed on Nov. 14, 2005.
(60) Provisional application No. 60/761,401, filed on Jan. 20, 2006.
(60) Provisional application No. 60/726,794, filed on Oct. 14, 2005.
(60) Provisional application No. 60/686,496, filed on May 31, 2005.
(60) Provisional application No. 60/422,007, filed on Oct. 29, 2002.
(60) Provisional application No. 60/732,413, filed on Nov. 1, 2005.
(60) Provisional application No. 60/736,961, filed on Nov. 14, 2005.
(60) Provisional application No. 60/761,404, filed on Jan. 23, 2006.
(60) Provisional application No. 60/945,570, filed on Jun. 21, 2007.
(60) Provisional application No. 60/951,707, filed on Jul. 24, 2007.
(60) Provisional application No. 60/968,043, filed on Aug. 24, 2007.
(60) Provisional application No. 61/018,303, filed on Dec. 31, 2007.
(60) Provisional application No. 60/951,707, filed on Jul. 24, 2007.
(60) Provisional application No. 60/968,043, filed on Aug. 24, 2007.
(60) Provisional application No. 61/018,303, filed on Dec. 31, 2007.
(60) Provisional application No. 60/951,707, filed on Jul. 24, 2007.
(60) Provisional application No. 60/968,043, filed on Aug. 24, 2007.
(60) Provisional application No. 61/018,303, filed on Dec. 31, 2007.
(60) Provisional application No. 60/951,707, filed on Jul. 24, 2007.
(60) Provisional application No. 60/799,455, filed on May 10, 2006.
(60) Provisional application No. 60/968,043, filed on Aug. 24, 2007.
(60) Provisional application No. 61/018,303, filed on Dec. 31, 2007.
(60) Provisional application No. 60/945,570, filed on Jun. 21, 2007.
(60) Provisional application No. 60/951,707, filed on Jul. 24, 2007.
(60) Provisional application No. 60/968,043, filed on Aug. 24, 2007.
(60) Provisional application No. 61/018,303, filed on Dec. 31, 2007.

(51) Int. Cl. A61B 17/22

(52) U.S. Cl. 606/159

(57) ABSTRACT

Embodiments are directed to devices for removing material from interior walls of vessels such as during atherectomy or thrombectomy procedures where the devices includes an ablation tool and at least one ablation tool stabilizer that can be used to radially position the ablation tool at desired locations within a vessel. In some embodiments, the ablation tool may a rotary cutting element that has an axis of rotation that is approximately parallel to the local axis of a vessel to be cleared. In some embodiments, the ablation tool may have a single side and or a top that allows clearing of material and which is capable of both radial positioning and rotational positioning via the stabilization device or devices and which may also be capable of axial motion via the stabilization device.

### RELATED APPLICATIONS

This application is a Continuation of U.S. patent application Ser. No. 12/203,126 (Microfabrica Docket No. P-US233-A-MF) which claims benefit of U.S. Provisional Patent Application Nos. 60/969,155 (P-US193-A-MF) and 61/034,916 (P-US203-A-MF), filed Mar. 7, 2008, and is a CIP of U.S. patent application Ser. No. 12/198,073 (P-US230-A-MF), filed Aug. 25, 2008, and U.S. patent application Ser. No. 12/179,573 (P-US225-A-MF), filed Jul. 24, 2008, and is a CIP of U.S. patent application Ser. No. 11/734,273 (P-US177-B-MF), filed Apr. 11, 2007. The '073 application in turn claims the benefit of U.S. Provisional Patent Application Nos. 60/968,030 (P-US190-A-MF), filed Aug. 24, 2007; and 60/968,006 (P-US191-A-MF), filed Aug. 24, 2007 and is a CIP of U.S. patent application Ser. No. 12/179,573 (P-US225-A-MF), filed Jul. 24, 2008, which claims benefit of U.S. Provisional Application Nos. 60/951,711 (P-US180-C-MF), filed Jul. 24, 2007; 60/968,042 (P-US180-D-MF), filed Aug. 24, 2007; and 61/018,283 (P-US180-E-MF), filed Dec. 31, 2007. The '573 application is a CIP of U.S. patent application Ser. Nos. 12/134,188 (P-US210-A-MF) filed Jun. 5, 2008; 11/625,807 (P-US171-A-MF), filed Jan. 22, 2007; 12/144,618 (P-US219-A-MF), filed Jun. 23, 2008; and 12/179,295 (P-US221-A-MF), filed Jul. 24, 2008. The '188 application in turn claims benefit to U.S. Provisional Application Nos. 60/942,200 (P-US178-A-MF), filed Jun. 5, 2007; 60/943,310 (P-US180-A-MF), filed Jun. 12, 2007; 60/949,850 (P-US180-B-MF), filed Jul. 14, 2007; 60/951,711 (P-US180-C-MF), filed Jul. 24, 2007; 60/968,042 (P-US180-D-MF), filed Aug. 24, 2007; 61/018,283 (P-US180-E-MF), filed Dec. 31, 2007; 60/945,570 (P-US185-A-MF), filed Jun. 21, 2007; 60/951,707 (P-US187-A-MF), filed Jul. 24, 2007; 60/968,043 (P-US189-A-MF), filed Aug. 24, 2007; and 61/018,303 (P-US189-A-MF), filed Dec. 31, 2007. The '126 application is a CIP of U.S. patent application Ser. No. 11/625,807 (P-US171-A-MF), filed Jan. 22, 2007. The '807 (171-A) application in turn claims benefit to U.S. Provisional Application No. 60/761,401 (P-US150-C-MF), filed Jan. 20, 2006, and is a CIP of U.S. application Ser. Nos. 11/598,968 (P-US167-A-MF), filed Nov. 14, 2006; 11/582,049 (P-US164-A-MF), filed Oct. 16, 2006; 11/444,999 (P-US159-A-MF), filed May 31, 2006; and 10/697,598 (P-US083-A-MG), filed Oct. 29, 2003. The '968 application claims benefit to U.S. Provisional Application Nos. 60/736,961 (P-US150-B-MF), filed Nov. 14, 2005, and 60/761,401 (P-US150-C-MF), filed Jan. 20, 2006, and is a CIP of U.S. patent application Ser. No. 11/591,911 (P-US165-A-MF), filed Nov. 1, 2006. The '049 application in turn claims the benefit to U.S. Provisional Patent Application No. 60/726,794 (P-US149-A-MF), filed Oct. 14, 2005. The '999 application claims benefit of U.S. Provisional Patent Application No. 60/686,496 (P-US145-A-MF), filed May 31, 2005 and is a CIP of U.S. patent application Ser. No. 10/697,598 (P-US083-A-MG), filed Oct. 29, 2003. The '598 application claims benefit of U.S. Provisional Patent Application No. 60/422,007 (P-US039-A-MG), filed Oct. 29, 2002. The '911 application claims benefit of U.S. Provisional Application Nos. 60/732,413 (P-US150-A-MF), filed Nov. 1, 2005; 60/736,961 (P-US150-B-MF), filed Nov. 14, 2005; and 60/761,401 (P-US150-C-MF), filed Jan. 20, 2006. The '618 application in turn claims benefit to U.S. Provisional Patent Application Nos. 60/945,570, (P-US185-A-MF), filed Jun. 21, 2007; 60/951,707, (P-US187-A-MF), filed Jul. 24, 2007; 60/968,043 (P-US189-A-MF), filed Aug. 24, 2007; and 61/018,303 (P-US189-B-MF), filed Dec. 31, 2007. The '295 application in turn is a CIP of U.S. patent application Ser. Nos. 12/169,528 (P-US220-A-MF), filed Jul. 8, 2008 and 12/144,618 (P-219-A-MF), filed Jun. 23, 2008 and claims benefit of U.S. Provisional Patent Application Nos. 60/951,707, (P-US187-A-MF), filed Jul. 24, 2007; 60/968,043 (P-US189-A-MF), filed Aug. 24, 2007; and 61/018,303 (P-US189-B-MF), filed Dec. 31, 2007. The '528 application in turn claims benefit of U.S. Provisional Patent Application Nos. 60/951,707, (P-US187-A-MF), filed Jul. 24, 2007; 60/968,043 (P-US189-A-MF), filed Aug. 24, 2007; and 61/018,303 (P-US189-B-MF), filed Dec. 31, 2007. The '528 application is a CIP of U.S. patent application Ser. No. 12/144,618 (P-US219-A-MF), filed Jun. 23, 2008 which in turn claims benefit of U.S. Provisional Patent Application Nos. 60/945,570, (P-US185-A-MF), filed Jun. 21, 2007; 60/951,707, (P-US187-A-MF), filed Jul. 24, 2007; 60/968,043 (P-US189-A-MF), filed Aug. 24, 2007; and 61/018,303 (P-US189-B-MF), filed Dec. 31, 2007. The '273 application in turn claims benefit of U.S. Provisional Patent Application Nos. 60/799,455 (P-US156-B-MF), filed May 10, 2008, and 60/790,917 (P-US156-A-MF), filed Apr. 11, 2006. Each of these applications is incorporated herein by reference as if set forth in full herein.

### FIELD OF THE INVENTION

The present invention relates devices for removal material from interior walls of a vessel and more particular to such devices that can be used in atherectomy or thrombectomy procedures. In some embodiments, such devices are fabricated at least in part using multi-layer, multi-material electrochemical fabrication methods.

### BACKGROUND OF THE INVENTION

### Electrochemical Fabrication

An electrochemical fabrication technique for forming three-dimensional structures from a plurality of adhered layers is being commercially pursued by Microfabrica® Inc. (formerly MEMGen Corporation) of Van Nuys, Calif. under the name EFAB®.

Various electrochemical fabrication techniques were described in U.S. Pat. No. 6,027,630, issued on Feb. 22, 2000 to Adam Cohen. Some embodiments of this electrochemical fabrication technique allows the selective deposition of a material using a mask that includes a patterned conformable material on a support structure that is independent of the substrate onto which plating will occur. When desiring to perform an electrodeposition using the mask, the conformable portion of the mask is brought into contact with a substrate, but not adhered or bonded to the substrate, while in the presence of a plating solution such that the contact of the conformable portion of the mask to the substrate inhibits deposition at selected locations. For convenience, these masks might be generically called conformable contact masks; the masking technique may be generically called a conformable contact mask plating process. More specifically, in the terminology of Microfabrica Inc. such masks have come to be known as INSTANT MASKS™ and the process known as INSTANT MASKING™ or INSTANT MASK™ plating. Selective depositions using conformable contact mask plating may be used to form single selective deposits of material or may be used in a process to form multi-layer structures. The teachings of the '630 patent are hereby incorporated herein by reference as if set forth in full herein. Since the filing of the patent application that led to the above noted patent, various papers about conformable contact mask plating (i.e. INSTANT MASKING) and electrochemical fabrication have been published:
(1) A. Cohen, G. Zhang, F. Tseng, F. Mansfeld, U. Frodis and P. Will, “EFAB: Batch production of functional, fully-dense metal parts with micro-scale features”, Proc. 9th Solid Freeform Fabrication, The University of Texas at Austin, p161, August 1998.
(2) A. Cohen, G. Zhang, F. Tseng, F. Mansfeld, U. Frodis and P. Will, “EFAB: Rapid, Low-Cost Desktop Micromachining of High Aspect Ratio True 3-D MEMS”, Proc. 12th IEEE Micro Electro Mechanical Systems Workshop, IEEE, p244, January 1999.
(3) A. Cohen, “3-D Micromachining by Electrochemical Fabrication”, Micromachine Devices, March 1999.
(4) G. Zhang, A. Cohen, U. Frodis, F. Tseng, F. Mansfeld, and P. Will, “EFAB: Rapid Desktop Manufacturing of True 3-D Microstructures”, Proc. 2nd International Conference on Integrated MicroNanotechnology for Space Applications, The Aerospace Co., April 1999.
(5) F. Tseng, U. Frodis, G. Zhang, A. Cohen, F. Mansfeld, and P. Will, “EFAB: High Aspect Ratio, Arbitrary 3-D Metal Microstructures using a Low-Cost Automated Batch Process”, 3rd International Workshop on High Aspect Ratio MicroStructure Technology (HARMST'99), June 1999.
(6) A. Cohen, U. Frodis, F. Tseng, G. Zhang, F. Mansfeld, and P. Will, “EFAB: Low-Cost, Automated Electrochemical Batch Fabrication of Arbitrary 3-D Microstructures”, Micromachining and Microfabrication Process Technology, SPIE 1999 Symposium on Micromachining and Microfabrication, September 1999.
(7) F. Tseng, G. Zhang, U. Frodis, A. Cohen, F. Mansfeld, and P. Will, “EFAB: High Aspect Ratio, Arbitrary 3-D Metal Microstructures using a Low-Cost Automated Batch Process”, MEMS Symposium, ASME 1999 International Mechanical Engineering Congress and Exposition, November, 1999.
(8) A. Cohen, “Electrochemical Fabrication (EFAB™)”, Chapter 19 of The MEMS Handbook, edited by Mohamed Gad-El-Hak, CRC Press, 2002.
(9) Microfabrication—Rapid Prototyping's Killer Application”, pages 1-5 of the Rapid Prototyping Report, CAD/CAM Publishing, Inc., June 1999.

The disclosures of these nine publications are hereby incorporated herein by reference as if set forth in full herein.

An electrochemical deposition for forming multilayer structures may be carried out in a number of different ways as set forth in the above patent and publications. In one form, this process involves the execution of three separate operations during the formation of each layer of the structure that is to be formed:
1. Selectively depositing at least one material by electrodeposition upon one or more desired regions of a substrate. Typically this material is either a structural material or a sacrificial material.
2. Then, blanket depositing at least one additional material by electrodeposition so that the additional deposit covers both the regions that were previously selectively deposited onto, and the regions of the substrate that did not receive any previously applied selective depositions. Typically this material is the other of a structural material or a sacrificial material.
3. Finally, planarizing the materials deposited during the first and second operations to produce a smoothed surface of a first layer of desired thickness having at least one region containing the at least one material and at least one region containing at least the one additional material.

After formation of the first layer, one or more additional layers may be formed adjacent to an immediately preceding layer and adhered to the smoothed surface of that preceding layer. These additional layers are formed by repeating the first through third operations one or more times wherein the formation of each subsequent layer treats the previously formed layers and the initial substrate as a new and thickening substrate.

Once the formation of all layers has been completed, at least a portion of at least one of the materials deposited is generally removed by an etching process to expose or release the three-dimensional structure that was intended to be formed. The removed material is a sacrificial material while the material that forms part of the desired structure is a structural material.

The preferred method of performing the selective electrodeposition involved in the first operation is by conformable contact mask plating. In this type of plating, one or more conformable contact (CC) masks are first formed. The CC masks include a support structure onto which a patterned conformable dielectric material is adhered or formed. The conformable material for each mask is shaped in accordance with a particular cross-section of material to be plated (the pattern of conformable material is complementary to the pattern of material to be deposited). At least one CC mask is used for each unique cross-sectional pattern that is to be plated.

The support for a CC mask is typically a plate-like structure formed of a metal that is to be selectively electroplated and from which material to be plated will be dissolved. In this typical approach, the support will act as an anode in an electroplating process. In an alternative approach, the support may instead be a porous or otherwise perforated material through which deposition material will pass during an electroplating operation on its way from a distal anode to a deposition surface. In either approach, it is possible for multiple CC masks to share a common support, i.e. the patterns of conformable dielectric material for plating multiple layers of material may be located in different areas of a single support structure. When a single support structure contains multiple plating patterns, the entire structure is referred to as the CC mask while the individual plating masks may be referred to as “submasks”. In the present application such a distinction will be made only when relevant to a specific point being made.

In preparation for performing the selective deposition of the first operation, the conformable portion of the CC mask is placed in registration with and pressed against a selected portion of (1) the substrate, (2) a previously formed layer, or (3) a previously deposited portion of a layer on which deposition is to occur. The pressing together of the CC mask and relevant substrate occur in such a way that all openings, in the conformable portions of the CC mask contain plating solution. The conformable material of the CC mask that contacts the substrate acts as a barrier to electrodeposition while the openings in the CC mask that are filled with electroplating solution act as pathways for transferring material from an anode (e.g. the CC mask support) to the non-contacted portions of the substrate (which act as a cathode during the plating operation) when an appropriate potential and/or current are supplied.

An example of a CC mask and CC mask plating are shown in FIGS. 1A-1C. FIG. 1A shows a side view of a CC mask 8 consisting of a conformable or deformable (e.g. elastomeric) insulator 10 patterned on an anode 12. The anode has two functions. One is as a supporting material for the patterned insulator 10 to maintain its integrity and alignment since the pattern may be topologically complex (e.g., involving isolated “islands” of insulator material). The other function is as an anode for the electroplating operation. FIG. 1A also depicts a substrate 6, separated from mask 8, onto which material will be deposited during the process of forming a layer. CC mask plating selectively deposits material 22 onto substrate 6 by simply pressing the insulator against the substrate then electrodepositing material through apertures 26a and 26b in the insulator as shown in FIG. 1B. After deposition, the CC mask is separated, preferably non-destructively, from the substrate 6 as shown in FIG. 10.

The CC mask plating process is distinct from a “through-mask” plating process in that in a through-mask plating process the separation of the masking material from the substrate would occur destructively. Furthermore in a through mask plating process, opening in the masking material are typically formed while the masking material is in contact with and adhered to the substrate. As with through-mask plating, CC mask plating deposits material selectively and simultaneously over the entire layer. The plated region may consist of one or more isolated plating regions where these isolated plating regions may belong to a single structure that is being formed or may belong to multiple structures that are being formed simultaneously. In CC mask plating as individual masks are not intentionally destroyed in the removal process, they may be usable in multiple plating operations.

Another example of a CC mask and CC mask plating is shown in FIGS. 1D-1G. FIG. 1D shows an anode 12′ separated from a mask 8′ that includes a patterned conformable material 10′ and a support structure 20. FIG. 1D also depicts substrate 6 separated from the mask 8′. FIG. 1E illustrates the mask 8′ being brought into contact with the substrate 6. FIG. 1F illustrates the deposit 22′ that results from conducting a current from the anode 12′ to the substrate 6. FIG. 1G illustrates the deposit 22′ on substrate 6 after separation from mask 8′. In this example, an appropriate electrolyte is located between the substrate 6 and the anode 12′ and a current of ions coming from one or both of the solution and the anode are conducted through the opening in the mask to the substrate where material is deposited. This type of mask may be referred to as an anodeless INSTANT MASK™ (AIM) or as an anodeless conformable contact (ACC) mask.

Unlike through-mask plating, CC mask plating allows CC masks to be formed completely separate from the substrate on which plating is to occur (e.g. separate from a three-dimensional (3D) structure that is being formed). CC masks may be formed in a variety of ways, for example, using a photolithographic process. All masks can be generated simultaneously, e.g. prior to structure fabrication rather than during it. This separation makes possible a simple, low-cost, automated, self-contained, and internally-clean “desktop factory” that can be installed almost anywhere to fabricate 3D structures, leaving any required clean room processes, such as photolithography to be performed by service bureaus or the like.

An example of the electrochemical fabrication process discussed above is illustrated in FIGS. 2A-2F. These figures show that the process involves deposition of a first material 2 which is a sacrificial material and a second material 4 which is a structural material. The CC mask 8, in this example, includes a patterned conformable material (e.g. an elastomeric dielectric material) 10 and a support 12 which is made from deposition material 2. The conformal portion of the CC mask is pressed against substrate 6 with a plating solution 14 located within the openings 16 in the conformable material 10. An electric current, from power supply 18, is then passed through the plating solution 14 via (a) support 12 which doubles as an anode and (b) substrate 6 which doubles as a cathode. FIG. 2A illustrates that the passing of current causes material 2 within the plating solution and material 2 from the anode 12 to be selectively transferred to and plated on the substrate 6. After electroplating the first deposition material 2 onto the substrate 6 using CC mask 8, the CC mask 8 is removed as shown in FIG. 2B. FIG. 2C depicts the second deposition material 4 as having been blanket-deposited (i.e. non-selectively deposited) over the previously deposited first deposition material 2 as well as over the other portions of the substrate 6. The blanket deposition occurs by electroplating from an anode (not shown), composed of the second material, through an appropriate plating solution (not shown), and to the cathode/substrate 6. The entire two-material layer is then planarized to achieve precise thickness and flatness as shown in FIG. 2D. After repetition of this process for all layers, the multi-layer structure 20 formed of the second material 4 (i.e. structural material) is embedded in first material 2 (i.e. sacrificial material) as shown in FIG. 2E. The embedded structure is etched to yield the desired device, i.e. structure 20, as shown in FIG. 2F.

Various components of an exemplary manual electrochemical fabrication system 32 are shown in FIGS. 3A-3C. The system 32 consists of several subsystems 34, 36, 38, and 40. The substrate holding subsystem 34 is depicted in the upper portions of each of FIGS. 3A-3C and includes several components: (1) a carrier 48, (2) a metal substrate 6 onto which the layers are deposited, and (3) a linear slide 42 capable of moving the substrate 6 up and down relative to the carrier 48 in response to drive force from actuator 44. Subsystem 34 also includes an indicator 46 for measuring differences in vertical position of the substrate which may be used in setting or determining layer thicknesses and/or deposition thicknesses. The subsystem 34 further includes feet 68 for carrier 48 which can be precisely mounted on subsystem 36.

The CC mask subsystem 36 shown in the lower portion of FIG. 3A includes several components: (1) a CC mask 8 that is actually made up of a number of CC masks (i.e. submasks) that share a common support/anode 12, (2) precision X-stage 54, (3) precision Y-stage 56, (4) frame 72 on which the feet 68 of subsystem 34 can mount, and (5) a tank 58 for containing the electrolyte 16. Subsystems 34 and 36 also include appropriate electrical connections (not shown) for connecting to an appropriate power source (not shown) for driving the CC masking process.

The blanket deposition subsystem 38 is shown in the lower portion of FIG. 3B and includes several components: (1) an anode 62, (2) an electrolyte tank 64 for holding plating solution 66, and (3) frame 74 on which feet 68 of subsystem 34 may sit. Subsystem 38 also includes appropriate electrical connections (not shown) for connecting the anode to an appropriate power supply (not shown) for driving the blanket deposition process.

The planarization subsystem 40 is shown in the lower portion of FIG. 3C and includes a lapping plate 52 and associated motion and control systems (not shown) for planarizing the depositions.

In addition to teaching the use of CC masks for electrodeposition purposes, the '630 patent also teaches that the CC masks may be placed against a substrate with the polarity of the voltage reversed and material may thereby be selectively removed from the substrate. It indicates that such removal processes can be used to selectively etch, engrave, and polish a substrate, e.g., a plaque.

The '630 patent further indicates that the electroplating methods and articles disclosed therein allow fabrication of devices from thin layers of materials such as, e.g., metals, polymers, ceramics, and semiconductor materials. It further indicates that although the electroplating embodiments described therein have been described with respect to the use of two metals, a variety of materials, e.g., polymers, ceramics and semiconductor materials, and any number of metals can be deposited either by the electroplating methods therein, or in separate processes that occur throughout the electroplating method. It indicates that a thin plating base can be deposited, e.g., by sputtering, over a deposit that is insufficiently conductive (e.g., an insulating layer) so as to enable subsequent electroplating. It also indicates that multiple support materials (i.e. sacrificial materials) can be included in the electroplated element allowing selective removal of the support materials.

The '630 patent additionally teaches that the electroplating methods disclosed therein can be used to manufacture elements having complex microstructure and close tolerances between parts. An example is given with the aid of FIGS. 14A-14E of that patent. In the example, elements having parts that fit with close tolerances, e.g., having gaps between about 1-5 um, including electroplating the parts of the device in an unassembled, preferably pre-aligned, state and once fabricated. In such embodiments, the individual parts can be moved into operational relation with each other or they can simply fall together. Once together the separate parts may be retained by clips or the like.

Another method for forming microstructures from electroplated metals (i.e. using electrochemical fabrication techniques) is taught in U.S. Pat. No. 5,190,637 to Henry Guckel, entitled “Formation of Microstructures by Multiple Level Deep X-ray Lithography with Sacrificial Metal layers”. This patent teaches the formation of metal structure utilizing through mask exposures. A first layer of a primary metal is electroplated onto an exposed plating base to fill a void in a photoresist (the photoresist forming a through mask having a desired pattern of openings), the photoresist is then removed and a secondary metal is electroplated over the first layer and over the plating base. The exposed surface of the secondary metal is then machined down to a height which exposes the first metal to produce a flat uniform surface extending across both the primary and secondary metals. Formation of a second layer may then begin by applying a photoresist over the first layer and patterning it (i.e. to form a second through mask) and then repeating the process that was used to produce the first layer to produce a second layer of desired configuration. The process is repeated until the entire structure is formed and the secondary metal is removed by etching. The photoresist is formed over the plating base or previous layer by casting and patterning of the photoresist (i.e. voids formed in the photoresist) are formed by exposure of the photoresist through a patterned mask via X-rays or UV radiation and development of the exposed or unexposed areas.

The '637 patent teaches the locating of a plating base onto a substrate in preparation for electroplating materials onto the substrate. The plating base is indicated as typically involving the use of a sputtered film of an adhesive metal, such as chromium or titanium, and then a sputtered film of the metal that is to be plated. It is also taught that the plating base may be applied over an initial layer of sacrificial material (i.e. a layer or coating of a single material) on the substrate so that the structure and substrate may be detached if desired. In such cases after formation of the structure the sacrificial material forming part of each layer of the structure may be removed along the initial sacrificial layer to free the structure. Substrate materials mentioned in the '637 patent include silicon, glass, metals, and silicon with protected semiconductor devices. A specific example of a plating base includes about 150 angstroms of titanium and about 300 angstroms of nickel, both of which are sputtered at a temperature of 160° C. In another example it is indicated that the plating base may consist of 150 angstroms of titanium and 150 angstroms of nickel where both are applied by sputtering.

Electrochemical Fabrication provides the ability to form prototypes and commercial quantities of miniature objects, parts, structures, devices, and the like at reasonable costs and in reasonable times. In fact, Electrochemical Fabrication is an enabler for the formation of many structures that were hitherto impossible to produce. Electrochemical Fabrication opens the spectrum for new designs and products in many industrial fields. Even though Electrochemical Fabrication offers this new capability and it is understood that Electrochemical Fabrication techniques can be combined with designs and structures known within various fields to produce new structures, certain uses for Electrochemical Fabrication provide designs, structures, capabilities and/or features not known or obvious in view of the state of the art.

A need exists in various fields for miniature devices having improved characteristics, reduced fabrication times, reduced fabrication costs, simplified fabrication processes, greater versatility in device design, improved selection of materials, improved material properties, more cost effective and less risky production of such devices, and/or more independence between geometric configuration and the selected fabrication process.

### SUMMARY OF THE INVENTION

It is an object of some embodiments of the invention to provide an improved method for forming multi-layer three-dimensional structures.

It is an object of some embodiments of the invention to provide a meso-scale or microscale device useful for removing unwanted material from the interior walls of a vessel.

It is an object of some embodiments of the invention to provide improved methods for removing material from the interior walls of a vessel

Other objects and advantages of various embodiments of the invention will be apparent to those of skill in the art upon review of the teachings herein. The various embodiments of the invention, set forth explicitly herein or otherwise ascertained from the teachings herein, may address one or more of the above objects alone or in combination, or alternatively may address some other object ascertained from the teachings herein. It is not necessarily intended that all objects be addressed by any single aspect of the invention even though that may be the case with regard to some aspects.

A first aspect of the invention provides a procedure for removing material from interior walls of a vessel without damaging the walls of the vessel in the region from which material is to be removed, comprising: (a) supplying a catheter; (b) supplying an removal tool and at least one radial stabilizer, wherein the radial stabilizer comprises a central body and a plurality of extendable elements that can contact the walls, wherein the removal tool is located beyond the distal end of the catheter, or can be made to extend from the distal end of the catheter, and wherein the removal tool and stabilizer are in a fixed or controllable position relative to one another; (c) inserting the catheter into the vessel of the patient such that the removal tool and stabilizer are located in proximity to a region of material to be removed; (d) expanding the at least one stabilizer to fix the radial position of the removal tool relative to the vessel walls; (e) activating the removal tool; (f) adjusting the radial position of the removal tool relative to the vessel walls via movement of the central body of the stabilizer relative to the walls of the vessel to bring the removal tool in contact with the material to be removed and to remove at least a portion of the material; (g) adjusting the radial position of the removal tool via movement of the stabilizer while the stabilizer is anchored so as to remove material and adjusting the axial position of the removal tool with or without the stabilizer being anchored so as to position the removal tool to remove further material; and (h) repeating the radial and axial movements of the removal tool to remove a desired quantity of material from the vessel.

A second aspect of the invention provides a procedure for removing material from interior walls of a vessel without damaging the walls of the vessel in the region from which material is to be removed, comprising: (a) supplying a catheter; (b) supplying an removal tool and at least one radial stabilizer, wherein the radial stabilizer comprises a central body and a plurality of extendable elements that can contact the walls, wherein the removal tool is located beyond the distal end of the catheter, or can be made to extend from the distal end of the catheter, and wherein the removal tool and stabilizer are in a fixed or controllable position relative to one another; (c) inserting the catheter into the vessel of the patient such that the removal tool and stabilizer are located in proximity to a region of material to be removed; (d) expanding the at least one stabilizer to fix the radial position of the removal tool relative to the vessel walls; (e) activating the removal tool; (f) adjusting the radial position of the removal tool relative to the vessel walls via pivoting a head of the tool relative to another portion of the tool such that a radial sweeping of the tool can occur so as to bring the removal tool in contact with the material to be removed and to remove at least a portion of the material; (g) adjusting the axial position of the removal tool; and (h) repeating the radial and axial movements of the removal tool to remove a desired quantity of material from the vessel.

Various embodiments directed to atherectomy or thrombectomy devices exist and may make use of a variety of device elements. Such device elements may, for example, include (1) a device head having one or more of (1a) one or more ablating tools (i.e. tools that may be used to cut, abrade, pulverize, and/or liquefy material to be removed from the interior walls of a vessel); (1b) one or more radial stabilizing and adjustment elements which are able to position and move a central body of the stabilizer relative to extended foot or pad portions of the stabilizer which anchor the stabilizer against the vessel walls; (2) a catheter from the distal end of which the device head extends or is made to extend prior to use; (3) a power source for operating the ablating tool; (4) a power source or mechanism for opening, closing, and adjusting, the extendible feet or pads of the stabilizer; (5) optionally one or more vacuum orifices and sources and/or other devices, for capturing and removing material that is separated from the vessel walls; (6) optionally one or more distal protection devices that may be used to capture and retain removed material that may otherwise flow down-stream from the removal site; optionally one or more visualization components, other transducers, or feedback elements for providing information to the operator to aid the operator in understanding what is occurring during the use of the device; and (7) optionally one or more control systems, e.g. computers and software, and associated feedback elements for providing automated operation of the device during use so that a plurality of the ablation devices, anchors, vacuum devices, and distal protection devices may controlled in a coordinated manner to ensure optimal removal, minimal operation time, maximal capture of removed material, and overall optimal completion of the procedure; and (8) optionally one or more injection heads or sprayers that may apply a desired drug, coating material to the de-plaqued surfaces of the vessel to, for example, aid in vessel recovery or minimization of thrombus formation resulting from damaged tissue or remnants of removed material.

Various embodiments of the invention directed to fabrication of atherectomy and/or thrombectomy devices form at least portions of those devices using a multi-layer multi-material electrochemical fabrication process that includes forming the devices or device portions with each successive layer comprising at least two materials, one of which is a structural material and the other of which is a sacrificial material, and wherein each successive layer defines a successive cross-section of the three-dimensional structure, and wherein the forming of each of the plurality of successive layers includes: (i) depositing a first of the at least two materials; (ii) depositing a second of the at least two materials; and (B) after the forming of the plurality of successive layers, separating at least a portion of the sacrificial material from the structural material to reveal the three-dimensional structure.

Various embodiments directed to using a thrombectomy or atherectomy devices, such as those having the elements set forth above, may include a variety of steps or operations including: (1) feeding a device ablation tool to a location within a vessel that is to undergo thrombus or plaque removal; (2) anchoring the ablation tool within the vessel via the expansion of one or more stabilization devices (e.g. multi-leg, multi-feet mechanical expansion elements that allow two-dimensional radial positioning of a central body portion, e.g. 3, 4 or more, positioning elements); (3) if necessary, adjusting the location of the ablation tool to a desired starting position; (4) optionally, deploying, e.g. opening, a distal protection device; (5) optionally, activating a material capture device; (6) activating the ablation tool; (7) moving the ablation tool relative to the stabilization device or devices in a controlled manner (e.g. along a selected radial or axial path) to ablate selected radial portions or axial portions of the thrombus or plaque; (8) if necessary, move the ablation head to a new location (e.g. along an unselected one of a radial or axial path) with or without unseating one or more the stabilization devices; (9) repeating the ablation of step (7) for a new series of locations; (8) if desired, repeat (8)-(9) one or more times to complete removal of all desired plaque or thrombus in the vessel; and (10) extract the ablation tool and other device elements from the vessel.

Other aspects of the invention will be understood by those of skill in the art upon review of the teachings herein. Other aspects of the invention may, for example, involve atherectomy devices, thrombectomy devices, methods for forming such devices, combinations of features taken from the various embodiments set forth herein as well as other configurations, structures, functional relationships, and processes that have not been specifically set forth in any single embodiment of the invention but whose combination would be apparent to those of ordinary skill in the art upon review of the teachings set forth herein.

### BRIEF DESCRIPTION OF THE DRAWINGS

FIGS. 1A-1C schematically depict side views of various stages of a CC mask plating process, while FIGS. 1D-G schematically depict a side views of various stages of a CC mask plating process using a different type of CC mask.

FIGS. 2A-2F schematically depict side views of various stages of an electrochemical fabrication process as applied to the formation of a particular structure where a sacrificial material is selectively deposited while a structural material is blanket deposited.

FIGS. 3A-3C schematically depict side views of various example subassemblies that may be used in manually implementing the electrochemical fabrication method depicted in FIGS. 2A-2F.

FIGS. 4A-4F schematically depict the formation of a first layer of a structure using adhered mask plating where the blanket deposition of a second material overlays both the openings between deposition locations of a first material and the first material itself

FIG. 4G depicts the completion of formation of the first layer resulting from planarizing the deposited materials to a desired level.

FIGS. 4H and 4I respectively depict the state of the process after formation of the multiple layers of the structure and after release of the structure from the sacrificial material.

FIGS. 5A-5F provide schematic illustrations of a process of a first embodiment for performing an atherectomy or thrombectomy.

FIGS. 6A-6L provide schematic illustrations of a process of a second embodiment for performing an atherectomy or thrombectomy.

FIGS. 7A-7L provide schematic illustrations of a process of a third embodiment for performing an atherectomy or thrombectomy.

FIGS. 8A-8L provide schematic illustrations of a process of a fourth embodiment for performing an atherectomy or thrombectomy.

FIGS. 9A-91 provide schematic illustrations of a process of a fifth embodiment for performing an atherectomy or thrombectomy.

FIGS. 10A-10C provide perspective views of an atherectomy or thrombectomy device according to a sixth embodiment of the invention.

FIGS. 11A-11C provide perspective views of an atherectomy or thrombectomy device according to a seventh embodiment of the invention.

FIGS. 12A-12C provide perspective views of an atherectomy or thrombectomy device according to an eighth embodiment of the invention.

FIGS. 13A-13D provide perspective views of an atherectomy or thrombectomy device according to a ninth embodiment of the invention.

### DETAILED DESCRIPTION OF PREFERRED EMBODIMENTS

### Electrochemical Fabrication in General

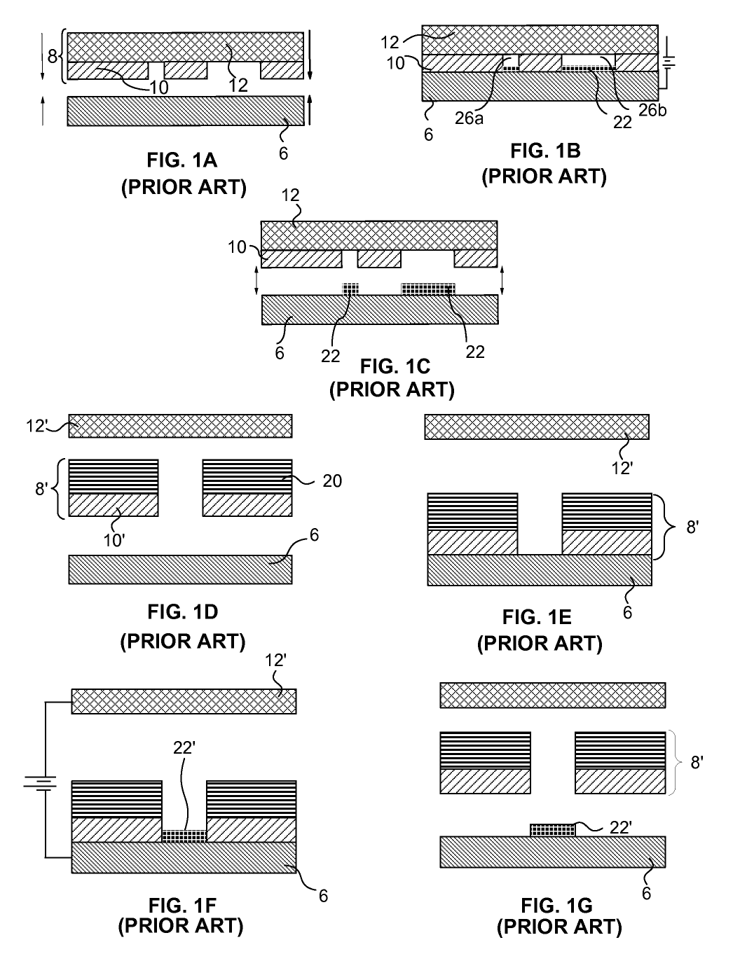

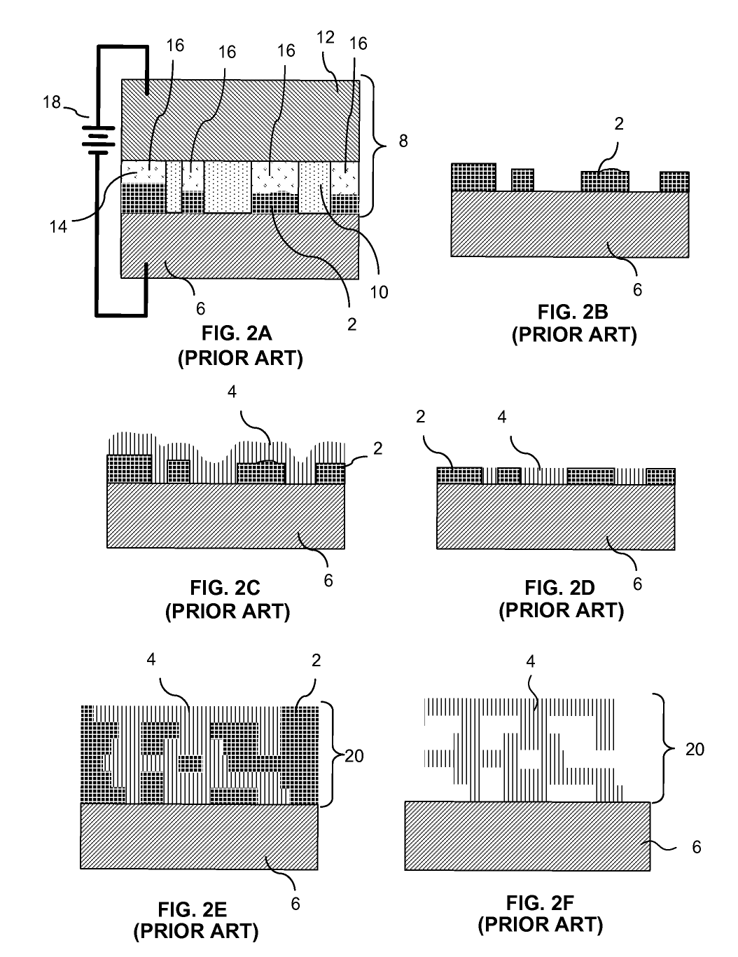

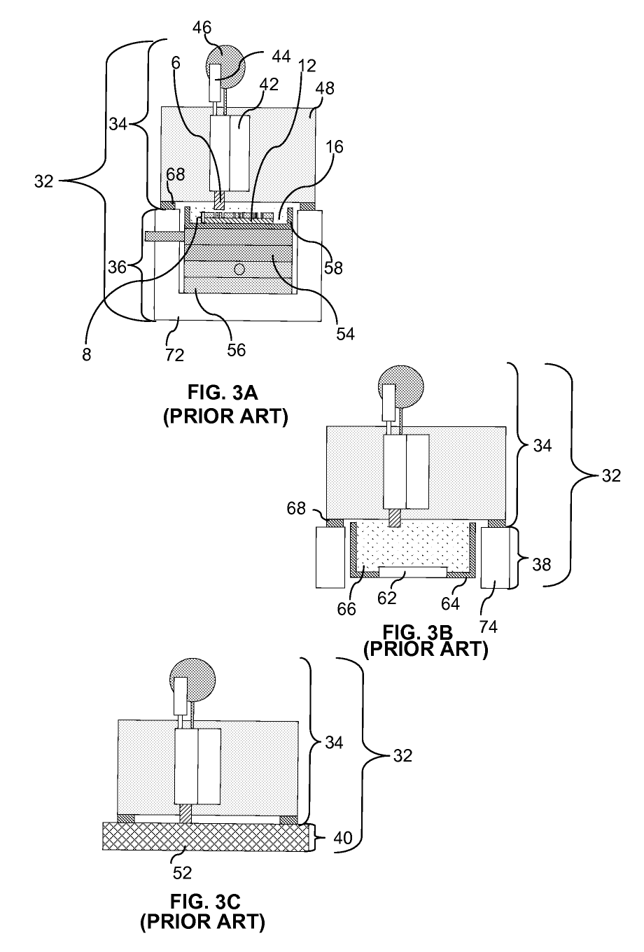

FIGS. 1A-1G, 2A-2F, and 3A-3C illustrate various features of one form of electrochemical fabrication. Other electrochemical fabrication techniques are set forth in the '630 patent referenced above, in the various previously incorporated publications, in various other patents and patent applications incorporated herein by reference. Still others may be derived from combinations of various approaches described in these publications, patents, and applications, or are otherwise known or ascertainable by those of skill in the art from the teachings set forth herein. All of these techniques may be combined with those of the various embodiments of various aspects of the invention to yield enhanced embodiments. Still other embodiments may be derived from combinations of the various embodiments explicitly set forth herein.

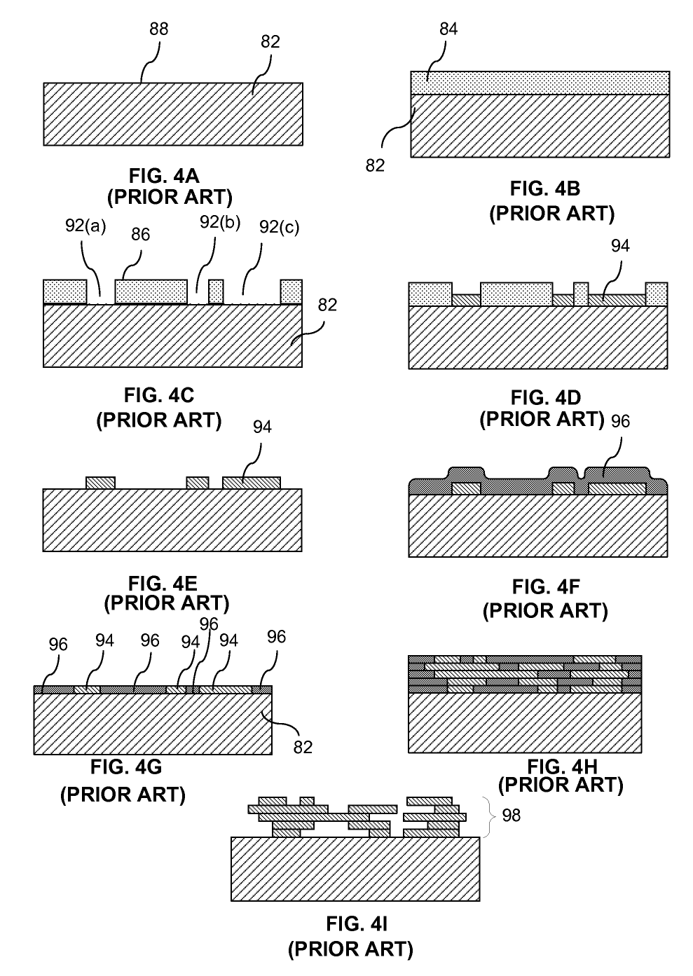

FIGS. 4A-4I illustrate various stages in the formation of a single layer of a multi-layer fabrication process where a second metal is deposited on a first metal as well as in openings in the first metal so that the first and second metal form part of the layer. In FIG. 4A a side view of a substrate 82 is shown, onto which patternable photoresist 84 is cast as shown in FIG. 4B. In FIG. 4C, a pattern of resist is shown that results from the curing, exposing, and developing of the resist. The patterning of the photoresist 84 results in openings or apertures 92(a)-92(c) extending from a surface 86 of the photoresist through the thickness of the photoresist to surface 88 of the substrate 82. In FIG. 4D a metal 94 (e.g. nickel) is shown as having been electroplated into the openings 92(a)-92(c). In FIG. 4E the photoresist has been removed (i.e. chemically stripped) from the substrate to expose regions of the substrate 82 which are not covered with the first metal 94. In FIG. 4F a second metal 96 (e.g. silver) is shown as having been blanket electroplated over the entire exposed portions of the substrate 82 (which is conductive) and over the first metal 94 (which is also conductive). FIG. 4G depicts the completed first layer of the structure which has resulted from the planarization of the first and second metals down to a height that exposes the first metal and sets a thickness for the first layer. In FIG. 4H the result of repeating the process steps shown in FIGS. 4B-4 G several times to form a multi-layer structure are shown where each layer consists of two materials. For most applications, one of these materials is removed as shown in FIG. 4I to yield a desired 3-D structure 98 (e.g. component or device).

Various embodiments of various aspects of the invention are directed to formation of three-dimensional structures from materials some of which may be electrodeposited or electroless deposited. Some of these structures may be formed form a single build level formed from one or more deposited materials while others are formed from a plurality of build layers each including at least two materials (e.g. two or more layers, more preferably five or more layers, and most preferably ten or more layers). In some embodiments, layer thicknesses may be as small as one micron or as large as fifty microns. In other embodiments, thinner layers may be used while in other embodiments, thicker layers may be used. In some embodiments structures having features positioned with micron level precision and minimum features size on the order of tens of microns are to be formed. In other embodiments structures with less precise feature placement and/or larger minimum features may be formed. In still other embodiments, higher precision and smaller minimum feature sizes may be desirable. In the present application meso-scale and millimeter scale have the same meaning and refer to devices that may have one or more dimensions extending into the 0.5-20 millimeter range, or somewhat larger and with features positioned with precision in the 10-100 micron range and with minimum features sizes on the order of 100 microns.

The various embodiments, alternatives, and techniques disclosed herein may form multi-layer structures using a single patterning technique on all layers or using different patterning techniques on different layers. For example, Various embodiments of the invention may perform selective patterning operations using conformable contact masks and masking operations (i.e. operations that use masks which are contacted to but not adhered to a substrate), proximity masks and masking operations (i.e. operations that use masks that at least partially selectively shield a substrate by their proximity to the substrate even if contact is not made), non-conformable masks and masking operations (i.e. masks and operations based on masks whose contact surfaces are not significantly conformable), and/or adhered masks and masking operations (masks and operations that use masks that are adhered to a substrate onto which selective deposition or etching is to occur as opposed to only being contacted to it). Conformable contact masks, proximity masks, and non-conformable contact masks share the property that they are preformed and brought to, or in proximity to, a surface which is to be treated (i.e. the exposed portions of the surface are to be treated). These masks can generally be removed without damaging the mask or the surface that received treatment to which they were contacted, or located in proximity to. Adhered masks are generally formed on the surface to be treated (i.e. the portion of that surface that is to be masked) and bonded to that surface such that they cannot be separated from that surface without being completely destroyed damaged beyond any point of reuse. Adhered masks may be formed in a number of ways including (1) by application of a photoresist, selective exposure of the photoresist, and then development of the photoresist, (2) selective transfer of pre-patterned masking material, and/or (3) direct formation of masks from computer controlled depositions of material.

Patterning operations may be used in selectively depositing material and/or may be used in the selective etching of material. Selectively etched regions may be selectively filled in or filled in via blanket deposition, or the like, with a different desired material. In some embodiments, the layer-by-layer build up may involve the simultaneous formation of portions of multiple layers. In some embodiments, depositions made in association with some layer levels may result in depositions to regions associated with other layer levels (i.e. regions that lie within the top and bottom boundary levels that define a different layer's geometric configuration). Such use of selective etching and interlaced material deposition in association with multiple layers is described in U.S. patent application Ser. No. 10/434,519, by Smalley, and entitled “Methods of and Apparatus for Electrochemically Fabricating Structures Via Interlaced Layers or Via Selective Etching and Filling of Voids layer elements” which is hereby incorporated herein by reference as if set forth in full.

Temporary substrates on which structures may be formed may be of the sacrificial-type (i.e. destroyed or damaged during separation of deposited materials to the extent they can not be reused), non-sacrificial-type (i.e. not destroyed or excessively damaged, i.e. not damaged to the extent they may not be reused, e.g. with a sacrificial or release layer located between the substrate and the initial layers of a structure that is formed). Non-sacrificial substrates may be considered reusable, with little or no rework (e.g. replanarizing one or more selected surfaces or applying a release layer, and the like) though they may or may not be reused for a variety of reasons.

### DEFINITIONS

This section of the specification is intended to set forth definitions for a number of specific terms that may be useful in describing the subject matter of the various embodiments of the invention. It is believed that the meanings of most if not all of these terms is clear from their general use in the specification but they are set forth hereinafter to remove any ambiguity that may exist. It is intended that these definitions be used in understanding the scope and limits of any claims that use these specific terms. As far as interpretation of the claims of this patent disclosure are concerned, it is intended that these definitions take presence over any contradictory definitions or allusions found in any materials which are incorporated herein by reference.

“Build” as used herein refers, as a verb, to the process of building a desired structure or plurality of structures from a plurality of applied or deposited materials which are stacked and adhered upon application or deposition or, as a noun, to the physical structure or structures formed from such a process. Depending on the context in which the term is used, such physical structures may include a desired structure embedded within a sacrificial material or may include only desired physical structures which may be separated from one another or may require dicing and/or slicing to cause separation.

“Build axis” or “build orientation” is the axis or orientation that is substantially perpendicular to substantially planar levels of deposited or applied materials that are used in building up a structure. The planar levels of deposited or applied materials may be or may not be completely planar but are substantially so in that the overall extent of their cross-sectional dimensions are significantly greater than the height of any individual deposit or application of material (e.g. 100, 500, 1000, 5000, or more times greater). The planar nature of the deposited or applied materials may come about from use of a process that leads to planar deposits or it may result from a planarization process (e.g. a process that includes mechanical abrasion, e.g. lapping, fly cutting, grinding, or the like) that is used to remove material regions of excess height. Unless explicitly noted otherwise, “vertical” as used herein refers to the build axis or nominal build axis (if the layers are not stacking with perfect registration) while “horizontal” refers to a direction within the plane of the layers (i.e. the plane that is substantially perpendicular to the build axis).

“Build layer” or “layer of structure” as used herein does not refer to a deposit of a specific material but instead refers to a region of a build located between a lower boundary level and an upper boundary level which generally defines a single cross-section of a structure being formed or structures which are being formed in parallel. Depending on the details of the actual process used to form the structure, build layers are generally formed on and adhered to previously formed build layers. In some processes the boundaries between build layers are defined by planarization operations which result in successive build layers being formed on substantially planar upper surfaces of previously formed build layers. In some embodiments, the substantially planar upper surface of the preceding build layer may be textured to improve adhesion between the layers. In other build processes, openings may exist in or be formed in the upper surface of a previous but only partially formed build layers such that the openings in the previous build layers are filled with materials deposited in association with current build layers which will cause interlacing of build layers and material deposits. Such interlacing is described in U.S. patent application Ser. No. 10/434,519. This referenced application is incorporated herein by reference as if set forth in full. In most embodiments, a build layer includes at least one primary structural material and at least one primary sacrificial material. However, in some embodiments, two or more primary structural materials may used without a primary sacrificial material (e.g. when one primary structural material is a dielectric and the other is a conductive material). In some embodiments, build layers are distinguishable from each other by the source of the data that is used to yield patterns of the deposits, applications, and/or etchings of material that form the respective build layers. For example, data descriptive of a structure to be formed which is derived from data extracted from different vertical levels of a data representation of the structure define different build layers of the structure. The vertical separation of successive pairs of such descriptive data may define the thickness of build layers associated with the data. As used herein, at times, “build layer” may be loosely referred simply as “layer”. In many embodiments, deposition thickness of primary structural or sacrificial materials (i.e. the thickness of any particular material after it is deposited) is generally greater than the layer thickness and a net deposit thickness is set via one or more planarization processes which may include, for example, mechanical abrasion (e.g. lapping, fly cutting, polishing, and the like) and/or chemical etching (e.g. using selective or non-selective etchants). The lower boundary and upper boundary for a build layer may be set and defined in different ways. From a design point of view they may be set based on a desired vertical resolution of the structure (which may vary with height). From a data manipulation point of view, the vertical layer boundaries may be defined as the vertical levels at which data descriptive of the structure is processed or the layer thickness may be defined as the height separating successive levels of cross-sectional data that dictate how the structure will be formed. From a fabrication point of view, depending on the exact fabrication process used, the upper and lower layer boundaries may be defined in a variety of different ways. For example by planarization levels or effective planarization levels (e.g. lapping levels, fly cutting levels, chemical mechanical polishing levels, mechanical polishing levels, vertical positions of structural and/or sacrificial materials after relatively uniform etch back following a mechanical or chemical mechanical planarization process). For example, by levels at which process steps or operations are repeated. At levels at which, at least theoretically, lateral extends of structural material can be changed to define new cross-sectional features of a structure.

“Layer thickness” is the height along the build axis between a lower boundary of a build layer and an upper boundary of that build layer.

“Planarization” is a process that tends to remove materials, above a desired plane, in a substantially non-selective manner such that all deposited materials are brought to a substantially common height or desired level (e.g. within 20%, 10%, 5%, or even 1% of a desired layer boundary level). For example, lapping removes material in a substantially non-selective manner though some amount of recession one material or another may occur (e.g. copper may recess relative to nickel). Planarization may occur primarily via mechanical means, e.g. lapping, grinding, fly cutting, milling, sanding, abrasive polishing, frictionally induced melting, other machining operations, or the like (i.e. mechanical planarization). Mechanical planarization maybe followed or proceeded by thermally induced planarization (e.g. melting) or chemically induced planarization (e.g. etching). Planarization may occur primarily via a chemical and/or electrical means (e.g. chemical etching, electrochemical etching, or the like). Planarization may occur via a simultaneous combination of mechanical and chemical etching (e.g. chemical mechanical polishing (CMP)).

“Structural material” as used herein refers to a material that remains part of the structure when put into use.

“Supplemental structural material” as used herein refers to a material that forms part of the structure when the structure is put to use but is not added as part of the build layers but instead is added to a plurality of layers simultaneously (e.g. via one or more coating operations that applies the material, selectively or in a blanket fashion, to a one or more surfaces of a desired build structure that has been released from a sacrificial material.

“Primary structural material” as used herein is a structural material that forms part of a given build layer and which is typically deposited or applied during the formation of that build layer and which makes up more than 20% of the structural material volume of the given build layer. In some embodiments, the primary structural material may be the same on each of a plurality of build layers or it may be different on different build layers. In some embodiments, a given primary structural material may be formed from two or more materials by the alloying or diffusion of two or more materials to form a single material.

“Secondary structural material” as used herein is a structural material that forms part of a given build layer and is typically deposited or applied during the formation of the given build layer but is not a primary structural material as it individually accounts for only a small volume of the structural material associated with the given layer. A secondary structural material will account for less than 20% of the volume of the structural material associated with the given layer. In some preferred embodiments, each secondary structural material may account for less than 10%, 5%, or even 2% of the volume of the structural material associated with the given layer. Examples of secondary structural materials may include seed layer materials, adhesion layer materials, barrier layer materials (e.g. diffusion barrier material), and the like. These secondary structural materials are typically applied to form coatings having thicknesses less than 2 microns, 1 micron, 0.5 microns, or even 0.2 microns). The coatings may be applied in a conformal or directional manner (e.g. via CVD, PVD, electroless deposition, or the like). Such coatings may be applied in a blanket manner or in a selective manner. Such coatings may be applied in a planar manner (e.g. over previously planarized layers of material) as taught in U.S. patent application Ser. No. 10/607,931. In other embodiments, such coatings may be applied in a non-planar manner, for example, in openings in and over a patterned masking material that has been applied to previously planarized layers of material as taught in U.S. patent application Ser. No. 10/841,383. These referenced applications are incorporated herein by reference as if set forth in full herein.

“Functional structural material” as used herein is a structural material that would have been removed as a sacrificial material but for its actual or effective encapsulation by other structural materials. Effective encapsulation refers, for example, to the inability of an etchant to attack the functional structural material due to inaccessibility that results from a very small area of exposure and/or due to an elongated or tortuous exposure path. For example, large (10,000 μm²) but thin (e.g. less than 0.5 microns) regions of sacrificial copper sandwiched between deposits of nickel may define regions of functional structural material depending on ability of a release etchant to remove the sandwiched copper.

“Sacrificial material” is material that forms part of a build layer but is not a structural material. Sacrificial material on a given build layer is separated from structural material on that build layer after formation of that build layer is completed and more generally is removed from a plurality of layers after completion of the formation of the plurality of layers during a “release” process that removes the bulk of the sacrificial material or materials. In general sacrificial material is located on a build layer during the formation of one, two, or more subsequent build layers and is thereafter removed in a manner that does not lead to a planarized surface. Materials that are applied primarily for masking purposes, i.e. to allow subsequent selective deposition or etching of a material, e.g. photoresist that is used in forming a build layer but does not form part of the build layer) or that exist as part of a build for less than one or two complete build layer formation cycles are not considered sacrificial materials as the term is used herein but instead shall be referred as masking materials or as temporary materials. These separation processes are sometimes referred to as a release process and may or may not involve the separation of structural material from a build substrate. In many embodiments, sacrificial material within a given build layer is not removed until all build layers making up the three-dimensional structure have been formed. Of course sacrificial material may be, and typically is, removed from above the upper level of a current build layer during planarization operations during the formation of the current build layer. Sacrificial material is typically removed via a chemical etching operation but in some embodiments may be removed via a melting operation or electrochemical etching operation. In typical structures, the removal of the sacrificial material (i.e. release of the structural material from the sacrificial material) does not result in planarized surfaces but instead results in surfaces that are dictated by the boundaries of structural materials located on each build layer. Sacrificial materials are typically distinct from structural materials by having different properties therefrom (e.g. chemical etchability, hardness, melting point, etc.) but in some cases, as noted previously, what would have been a sacrificial material may become a structural material by its actual or effective encapsulation by other structural materials. Similarly, structural materials may be used to form sacrificial structures that are separated from a desired structure during a release process via the sacrificial structures being only attached to sacrificial material or potentially by dissolution of the sacrificial structures themselves using a process that is insufficient to reach structural material that is intended to form part of a desired structure. It should be understood that in some embodiments, small amounts of structural material may be removed, after or during release of sacrificial material. Such small amounts of structural material may have been inadvertently formed due to imperfections in the fabrication process or may result from the proper application of the process but may result in features that are less than optimal (e.g. layers with stairs steps in regions where smooth sloped surfaces are desired. In such cases the volume of structural material removed is typically minuscule compared to the amount that is retained and thus such removal is ignored when labeling materials as sacrificial or structural. Sacrificial materials are typically removed by a dissolution process, or the like, that destroys the geometric configuration of the sacrificial material as it existed on the build layers. In many embodiments, the sacrificial material is a conductive material such as a metal. As will be discussed hereafter, masking materials though typically sacrificial in nature are not termed sacrificial materials herein unless they meet the required definition of sacrificial material.

“Supplemental sacrificial material” as used herein refers to a material that does not form part of the structure when the structure is put to use and is not added as part of the build layers but instead is added to a plurality of layers simultaneously (e.g. via one or more coating operations that applies the material, selectively or in a blanket fashion, to a one or more surfaces of a desired build structure that has been released from an initial sacrificial material. This supplemental sacrificial material will remain in place for a period of time and/or during the performance of certain post layer formation operations, e.g. to protect the structure that was released from a primary sacrificial material, but will be removed prior to putting the structure to use.

“Primary sacrificial material” as used herein is a sacrificial material that is located on a given build layer and which is typically deposited or applied during the formation of that build layer and which makes up more than 20% of the sacrificial material volume of the given build layer. In some embodiments, the primary sacrificial material may be the same on each of a plurality of build layers or may be different on different build layers. In some embodiments, a given primary sacrificial material may be formed from two or more materials by the alloying or diffusion of two or more materials to form a single material.

“Secondary sacrificial material” as used herein is a sacrificial material that is located on a given build layer and is typically deposited or applied during the formation of the build layer but is not a primary sacrificial materials as it individually accounts for only a small volume of the sacrificial material associated with the given layer. A secondary sacrificial material will account for less than 20% of the volume of the sacrificial material associated with the given layer. In some preferred embodiments, each secondary sacrificial material may account for less than 10%, 5%, or even 2% of the volume of the sacrificial material associated with the given layer. Examples of secondary structural materials may include seed layer materials, adhesion layer materials, barrier layer materials (e.g. diffusion barrier material), and the like. These secondary sacrificial materials are typically applied to form coatings having thicknesses less than 2 microns, 1 micron, 0.5 microns, or even 0.2 microns). The coatings may be applied in a conformal or directional manner (e.g. via CVD, PVD, electroless deposition, or the like). Such coatings may be applied in a blanket manner or in a selective manner. Such coatings may be applied in a planar manner (e.g. over previously planarized layers of material) as taught in U.S. patent application Ser. No. 10/607,931. In other embodiments, such coatings may be applied in a non-planar manner, for example, in openings in and over a patterned masking material that has been applied to previously planarized layers of material as taught in U.S. patent application Ser. No. 10/841,383. These referenced applications are incorporated herein by reference as if set forth in full herein.

“Adhesion layer”, “seed layer”, “barrier layer”, and the like refer to coatings of material that are thin in comparison to the layer thickness and thus generally form secondary structural material portions or sacrificial material portions of some layers. Such coatings may be applied uniformly over a previously formed build layer, they may be applied over a portion of a previously formed build layer and over patterned structural or sacrificial material existing on a current (i.e. partially formed) build layer so that a non-planar seed layer results, or they may be selectively applied to only certain locations on a previously formed build layer. In the event such coatings are non-selectively applied, selected portions may be removed (1) prior to depositing either a sacrificial material or structural material as part of a current layer or (2) prior to beginning formation of the next layer or they may remain in place through the layer build up process and then etched away after formation of a plurality of build layers.

“Masking material” is a material that may be used as a tool in the process of forming a build layer but does not form part of that build layer. Masking material is typically a photopolymer or photoresist material or other material that may be readily patterned. Masking material is typically a dielectric. Masking material, though typically sacrificial in nature, is not a sacrificial material as the term is used herein. Masking material is typically applied to a surface during the formation of a build layer for the purpose of allowing selective deposition, etching, or other treatment and is removed either during the process of forming that build layer or immediately after the formation of that build layer.

“Multilayer structures” are structures formed from multiple build layers of deposited or applied materials.

“Multilayer three-dimensional (or 3D or 3-D) structures” are Multilayer Structures that meet at least one of two criteria: (1) the structural material portion of at least two layers of which one has structural material portions that do not overlap structural material portions of the other.

“Complex multilayer three-dimensional (or 3D or 3-D) structures” are multilayer three-dimensional structures formed from at least three layers where a line may be defined that hypothetically extends vertically through at least some portion of the build layers of the structure will extend from structural material through sacrificial material and back through structural material or will extend from sacrificial material through structural material and back through sacrificial material (these might be termed vertically complex multilayer three-dimensional structures). Alternatively, complex multilayer three-dimensional structures may be defined as multilayer three-dimensional structures formed from at least two layers where a line may be defined that hypothetically extends horizontally through at least some portion of a build layer of the structure that will extend from structural material through sacrificial material and back through structural material or will extend from sacrificial material through structural material and back through sacrificial material (these might be termed horizontally complex multilayer three-dimensional structures). Worded another way, in complex multilayer three-dimensional structures, a vertically or horizontally extending hypothetical line will extend from one or structural material or void (when the sacrificial material is removed) to the other of void or structural material and then back to structural material or void as the line is traversed along at least a portion of the line.

“Moderately complex multilayer three-dimensional (or 3D or 3-D) structures are complex multilayer 3D structures for which the alternating of void and structure or structure and void not only exists along one of a vertically or horizontally extending line but along lines extending both vertically and horizontally.

“Highly complex multilayer (or 3D or 3-D) structures are complex multilayer 3D structures for which the structure-to-void-to-structure or void-to-structure-to-void alternating occurs once along the line but occurs a plurality of times along a definable horizontally or vertically extending line.

“Up-facing feature” is an element dictated by the cross-sectional data for a given build layer “n” and a next build layer “n+1” that is to be formed from a given material that exists on the build layer “n” but does not exist on the immediately succeeding build layer “n+1”. For convenience the term “up-facing feature” will apply to such features regardless of the build orientation.

“Down-facing feature” is an element dictated by the cross-sectional data for a given build layer “n” and a preceding build layer “n−1” that is to be formed from a given material that exists on build layer “n” but does not exist on the immediately preceding build layer “n−1”. As with up-facing features, the term “down-facing feature” shall apply to such features regardless of the actual build orientation.

“Continuing region” is the portion of a given build layer “n” that is dictated by the cross-sectional data for the given build layer “n”, a next build layer “n+1” and a preceding build layer “n−1” that is neither up-facing nor down-facing for the build layer “n”.

“Minimum feature size” refers to a necessary or desirable spacing between structural material elements on a given layer that are to remain distinct in the final device configuration. If the minimum feature size is not maintained on a given layer, the fabrication process may result in structural material inadvertently bridging the two structural elements due to masking material failure or failure to appropriately fill voids with sacrificial material during formation of the given layer such that during formation of a subsequent layer structural material inadvertently fills the void. More care during fabrication can lead to a reduction in minimum feature size or a willingness to accept greater losses in productivity can result in a decrease in the minimum feature size. However, during fabrication for a given set of process parameters, inspection diligence, and yield (successful level of production) a minimum design feature size is set in one way or another. The above described minimum feature size may more appropriately be termed minimum feature size of sacrificial material regions. Conversely a minimum feature size for structure material regions (minimum width or length of structural material elements) may be specified. Depending on the fabrication method and order of deposition of structural material and sacrificial material, the two types of minimum feature sizes may be different. In practice, for example, using electrochemical fabrication methods and described herein, the minimum features size on a given layer may be roughly set to a value that approximates the layer thickness used to form the layer and it may be considered the same for both structural and sacrificial material widths and lengths. In some more rigorously implemented processes, examination regiments, and rework requirements, it may be set to an amount that is 80%, 50%, or even 30% of the layer thickness. Other values or methods of setting minimum feature sizes may be set.

“Sublayer” as used herein refers to a portion of a build layer that typically includes the full lateral extents of that build layer but only a portion of its height. A sublayer is usually a vertical portion of build layer that undergoes independent processing compared to another sublayer of that build layer.

Atherectomy and Thrombectomy Devices and Methods of Use

Various embodiments of the device aspects of the invention exist and may make use of a variety of device elements including those listed above in the summary of the invention section of the application. In the various embodiments of the invention it is preferred, though not required, that the ablation tool take the form of a rotating element that spins along an axis parallel to the “local” axis of the vessel (i.e. local z-axis, or longitudinal axis). In such embodiments the rotating element preferably has a diameter that is small compared to the diameter of the vessel (e.g. less than ½ the local diameter of the vessel and more preferably less than ⅕ the diameter of the vessel). The effective cutting height of the rotating element is preferably comparable or less than its diameter. The rotating element, for example, may take an overall cylindrically shape or a tapering shape that decreases in diameter with distance from the supporting stabilizing element. The rotating element may include one or more radially extending blades, a grinding surface, a series of unsharpened paddles or spoons, blades or paddles that are tapped along the axial direction to provide removed material with a desired proximal or distal movement that may be directed toward a removal orifice. The blades or grinding surfaces may have fixed or variable radial extensions. The vessel walls may be protected from the cutting blades or abrasive surface by a cap or shield on the opposite side of the cutting elements relative to the stabilizer support. This may be particularly useful in embodiments where the ablation tool is not supported by a distal stabilizer and the ablation is occurring from a proximal position to a more distal position. In some embodiments, such protective elements may include a cylindrical cage-like element attached to the central body of the stabilizer, or to the stabilizer side of the ablation device, which extends more radially than the cutting elements and partially or fully along the axial height of the cutting elements and inhibits the cutting elements from directly contacting the vessel walls but not from contacting plaque that is either pushed into openings in the cage or that is displaced from the region protected by the cage. In some embodiments, the ablation tool may only provide material removal along its periphery (e.g. the outer most radial portions of the device) while in other embodiments it may also provide removal via the end that is away from or facing a stabilizing support (e.g. which would allow removal of material in an axial direction via interaction with cutting surfaces on the top of the cutter).

In the most preferred embodiments, though not all embodiments, that use mechanical machining techniques to remove plaque or thrombus, the cutting force that the removal tool exerts on the plaque or vessel side walls, or cutting location operated on by the tool, is preferably achieved by directly applied pressure or positioning set by the relationship between the positioning of the cutting tool and the central body portion of the stabilizer or stabilizers (e.g. from the X&Y adjustment of central body portion which moves the cutting tool to known locations) instead of via centrifugal force which is used to move elongated cutting elements into contact with plaque or vessel side walls at relative uncontrolled locations (i.e. possibly uncontrolled along the axis of the vessel and certainly uncontrolled along the sidewalls at different angular positions.

In some alternative embodiments, the ablation tool may take the form of a fixed or directable nozzle that is capable of directing a stream of water or other fluid at the plaque or thrombus to break it up. In some such embodiments, the nozzle may be located some distance from the stabilizer (e.g. distal to it) while its spray is directed at an angle back toward the expander and in particular toward one or more vacuum traps (e.g. vacuum trap) that are located near the inner walls of the vessel.

In different embodiments, the ablation tool may be supported from its proximal end via a fixed or variable length linkage to a proximal stabilizer, from its distal end via a fixed or variable length linkage to a distal stabilizer, or from both via a fixed or variable length linkage.

When in use, the one or more stabilizers of (1b) may be located (a) proximally and in proximity to a region along the vessel (z-axis) that is to undergo an atherectomy or thrombectomy; (b) distally in proximity to a region along the vessel (z-axis) that is to undergo an atherectomy or thrombectomy; (c) in both proximal and distal positions; (d) in the region containing material to be removed; and (e) in a selected one of the above noted locations while one or more additional stabilizers may be located in a different one of the above noted locations.

The positions of the body portion of the stabilizer of (1b) relative to the ablating tool (1a) may be fixed or adjustable in the radial direction (fixed is preferred but not required), and may be fixed or adjustable in an axial direction.

In some embodiments, a rotary ablation tool is powered locally while in other embodiments the rotary motion is provide via a cable that extends the length of the catheter. In locally powered embodiments, the rotary ablation tool may be attached to a turbine which is spun by fluid flow supplied up and/or down the catheter. In some turbine or fluid flow actuated embodiments, the drive fluid may be feed directly out of the body via the catheter without very being freely located within the vessel while in other embodiments the fluid may be dispensed into the vessel after imparting its actuation force and may then be extracted from the vessel. removed from the vessel. In some embodiments, a miniature electric motor may be used to provide rotary force.

Various embodiments directed to using a thrombectomy or atherectomy device, such as those having the elements set forth above, may include a variety of steps or operations including those as noted above in the summary of the invention section of the application.

During use, in some embodiments, the ablation tool may be made to clear a radial regions at a given longitudinal, axial, or z-axis position before being moved to a more distal or proximal position along the vessel after which another radial clearing at the new Z-axis position could occur.

When a device has two or more stabilizers, the stabilizers and ablation tool may be moved along the length of the vessel (i.e. z-axis) by disengaging both stabilizers from the vessel walls and translating the entirety to a new position along the vessel. Alternatively, when the position between the two stabilizers is not fixed, the stabilizers may be stepped or walked along the vessel, for example by un-anchoring one stabilizer and moving it in a desired direction (distally or proximally) while the other stabilizer remains anchored, after which the first stepped stabilizer is re-anchored in its new location and the other is disengaged and moved in the desired direction. In some embodiments, the use of a pair of stabilizers may aid in the feeding of the catheter along the length of a vessel.

In some embodiments, the stabilizers may provide force feedback to the operator or control system to ensure that the stabilizer is properly anchored without putting undue burden on the vessel walls. In some preferred embodiments, the stabilizer(s) may provide temporary forced assurance of the circularity of the vessel interior so that the ablation tool can operate in a path that provides optimal removal of material with minimal risk of damaging vessel walls as may occur if the movement of the ablator assumes a particular vessel wall configuration that is larger than exists in actuality.

In some preferred embodiments, the ablation tool may be limited to traverse a path that is within a polygon outlined by the pads of the stabilizer (e.g. a triangle if three pads are used, a quadrilateral if four pads are used, and the like).

In some embodiments, the stabilizer devices may be configured to provide assurance of known orientation relative to the axis of a vessel so that XY movement of an ablation tool can be assured to be within a desired tolerance of the XY plane of the vessel. In some implementations such assurance may be obtained by use of a stabilizer whose pads are long compared to the vessel diameter. Alternatively, it may be obtained by use of two stabilizers that are spaced from one another by a distance that is large compared to the vessel diameter and connected to one another by a resilient linkage. The two stabilizers may be opened little-by-little in alternating turns such that any disorientation is removed by one side of one touching a side wall first while the other side of the other touches the opposite side wall first with both contact points driving the pair of stabilizers to an more axial orientation. It may be possible to obtain a similar orientation by opening each stabilizer to anchor it fully then retracting slightly and then re-anchoring.

In some embodiments, it is preferred, but not required, that the removal occur in full radial cross-sections prior to elongating the cleaned regions axially. In such embodiments, it is preferable, but not required, to begin removal with the ablation tool located in a known or suspected opening in the plaque, e.g. near the vessel center, and then spiraling with progressively enlarging paths until tool proximity to the vessel walls is reached, proximity to the estimated positions of the vessel walls is reached, or the radial extent of a “safe zone” is reached (i.e. one that is considered safe for ablation tool operation without risking significant damage to or puncturing of vessel walls). In some such cases, the spiraling may be circular, elliptical, square, triangular, or the like or a version of one of these. In some cases the offset of successive spiral paths may not be uniform, e.g. when a starting location is not centered within a vessel. In other embodiments, the removal paths may be more like a series of parallel raster lines that extend from side-wall-to-side-wall. In still other embodiments, removal paths may be a series of ever enlarging polygonal paths. In still other embodiments, the paths may be dictated and limited to known plaque locations.

In some embodiments, movement of the cutting tool axially either by extending it from a fixed stabilizer or by moving the stabilizer axially occurs in a manner that minimizes the risk of perforating a wall of the vessel that may be turning in front of the cutting element. In some embodiments, the maximum amount of allowed axial walking movement is derived from known or anticipated curvature of the particular vessel or vessel type being acted upon in combination with a given confidence in the original orientation of the stabilizer(s).

Most preferred embodiments of the invention, though not necessarily all, address one or more of the issues existing with current atherectomy or thrombectomy procedures as the case maybe, for example (1) improved uniformity in cross-sectional (i.e. perpendicular to the vessel axis) cleaning may be provided; (2) reduced need to remove and clean the cutting tool may occur; (3) improved tolerance for variations in vessel diameter by a single tool may exist; and (4) reduced need for visualization for process success. In some embodiments, each of these improvements will be achieved while in others only a portion of them will be achieved.

In some embodiments, a cutting tool may start from a more proximal position and incrementally move toward a more distal position along the length of a vessel. In other embodiments, the cutting tool may be passed through a vessel region constricted with plaque and be operated to remove more distal plaque followed by the incremental removal of more proximal regions of plaque (which may allow removal to occur with less risk of causing vessel damage or puncture.

In some embodiments the device may include a guidewire or linkage that extends through the end of the ablation tool whether the tool be located in a distally facing manner or in a proximally facing manner such that other tools or device components may be located on the distal side of the cutter without impacting the ability of the cutter to access all radial regions of a vessel to be cleaned. In some embodiments, guide wires or linkages may exist alongside the ablation tool and simply be rotated out of the cutting path of the ablation tool (e.g. via a rotational motion of the bodies of the expansions tools along with a rotational motion of the ablation tool.

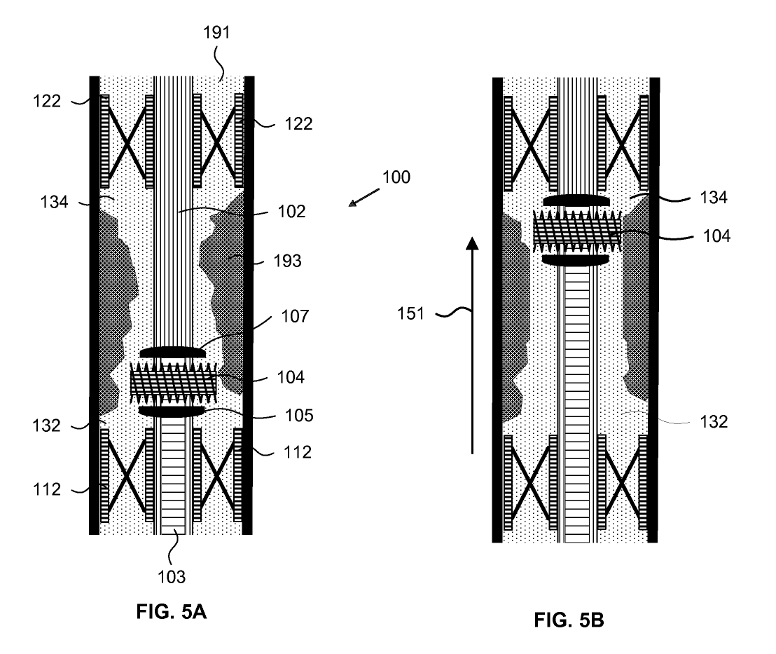

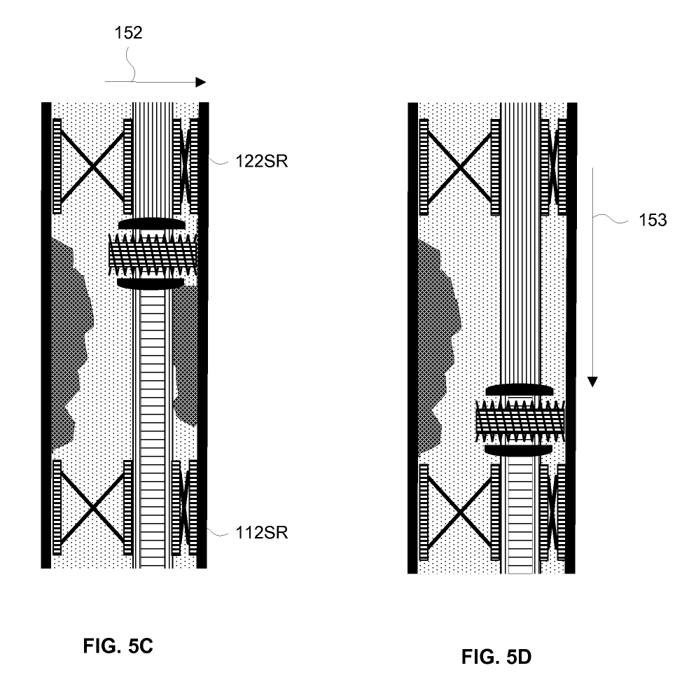

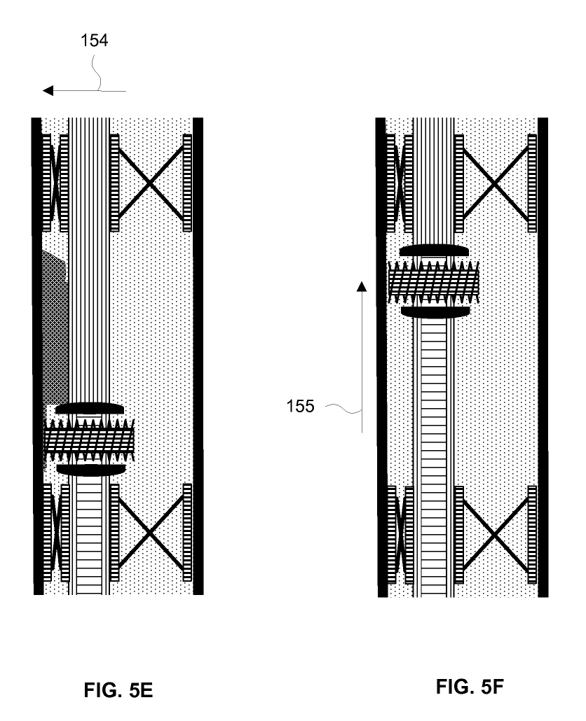

FIGS. 5A-5F provide schematic illustrations of a first process of a first embodiment for performing an atherectomy or thrombectomy using an atherectomy device or thrombectomy device 100 having an ablation device 104 and a first stabilization element 112 located on the proximal side 132 of the an obstruction 193 within a vessel 191 and a second stabilization device 122 located on the distal side 134 of the obstruction and where the device 100 is made to clean an axial strip followed by a radial increment to position the ablation tool for clearing a subsequent axial strip where the axial and radial movements are repeated a number of times until the portion of vessel is cleared. The starting position for the exemplary process is shown in FIG. 5A with subsequent steps shown in subsequent FIGS where motions in directions 151, 152, 153, 154, and 155 are taken in sequence. The ablation device may include upper and lower shields 105 and 107 and may be moved and operated relative to the vessel via a catheter or guide wire 102 and a control element 103. As illustrated only radial increments along the plane of the page (e.g. X-axis) are illustrated but in actual practice it would be desirable for radial sweeping to occur in an out of the page as well (e.g. Y-axis). In this embodiment, axial removal occurs during both proximal sweeps 153 and distal sweeps 151 and 155. In this embodiment, guide wires and/or linkages connect the proximal and distal stabilization devices and extend through the central portion of the ablation tool to aid in guidance of the ablation tool and to pass control signals to the distal stabilization elements 122 and to/or from any other distal elements. In this embodiment, for illustrative purposes the vessel is considered to be cleared after only left and right radial transitions and associated axial sweeps but in a vessel having a y-dimension (i.e. in and out of the page) that is greater than the diameter of the ablation tool, additional radial increments will occur if it is desired to further clear the vessel. In some alternative embodiments, both expansion devices may be located on the same side (e.g. the proximal side) of the obstruction. In some alternative embodiments, axial clearing may occur only during one of distal motion or proximal motion.

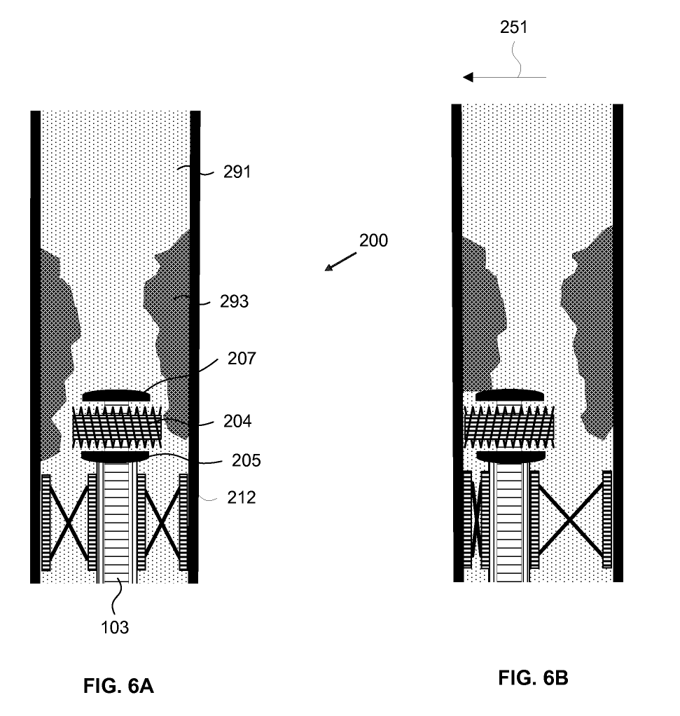

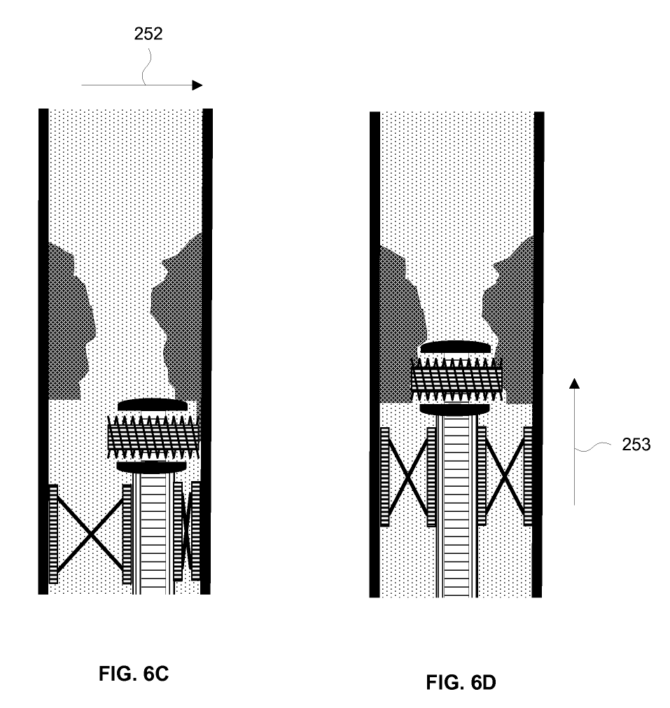

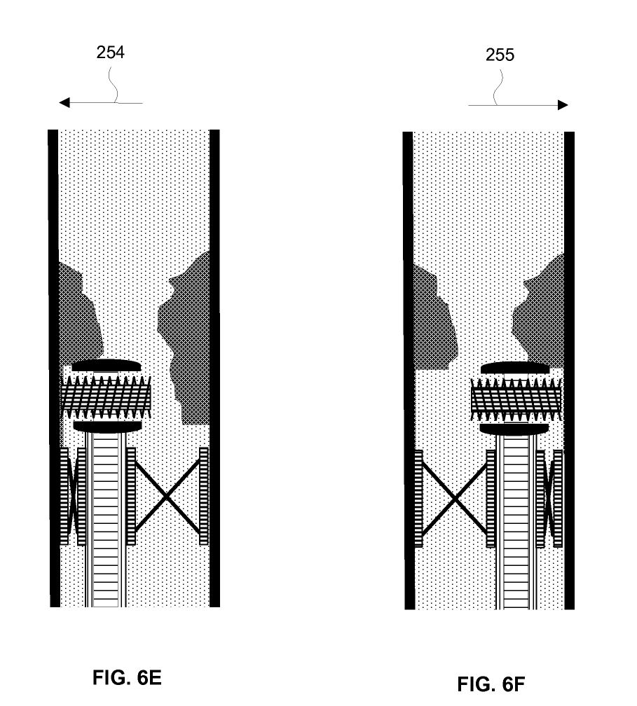

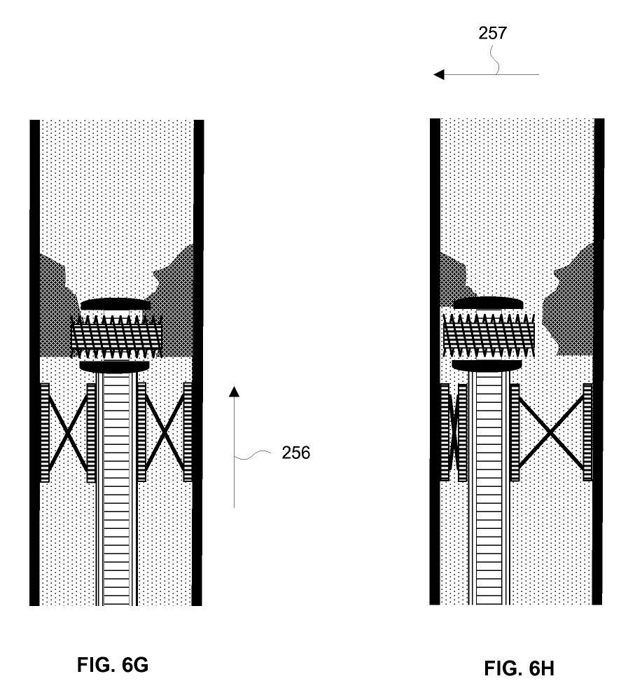

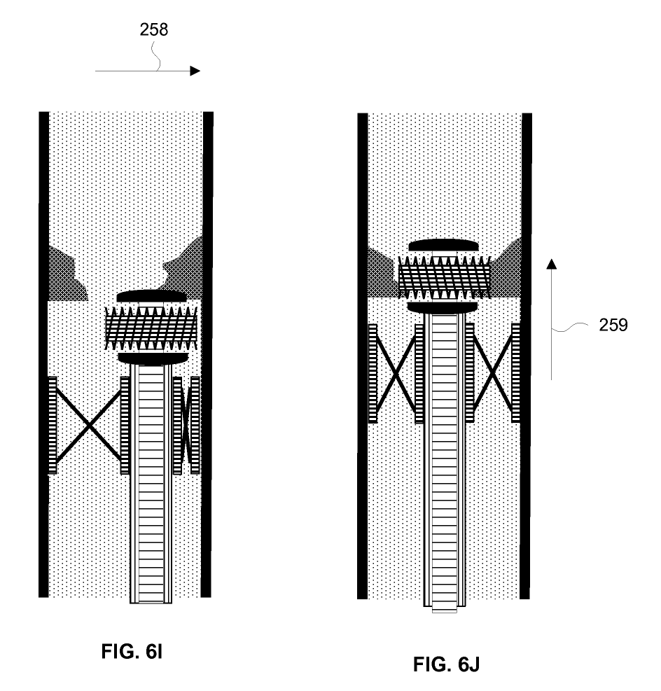

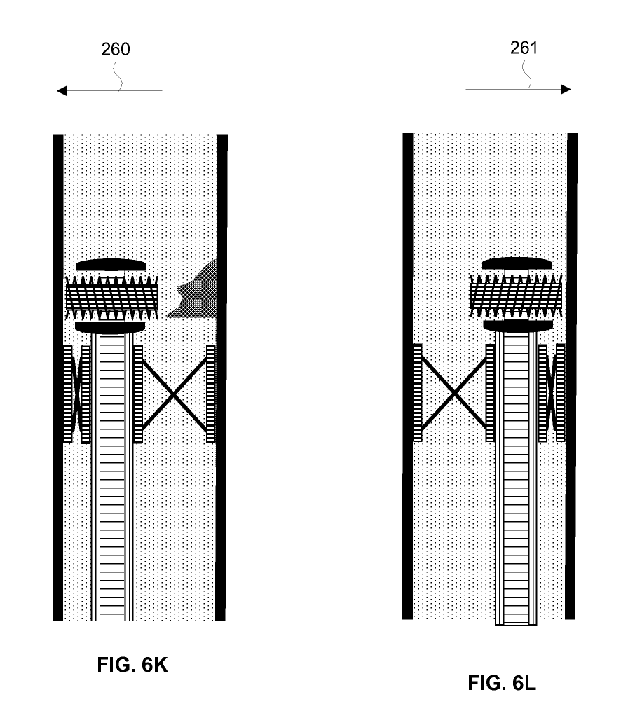

FIGS. 6A-6L provide schematic illustrations of a second process for performing an atherectomy or thrombectomy using an atherectomy device or thrombectomy device 200 having an ablation device 204, with optional proximal and distal caps 205 and 207 and a first stabilization element 212 located on the proximal side of the an obstruction 293 in vessel 291 and where the device is made to clean a radial cross-section of the vessel followed by stopping motion of the ablation device, release of the anchoring of the stabilization device, distal incrementing of the stabilization device 212, and re-anchoring of the stabilization device in proximity to the remaining obstruction, and then repeating the radial and axial movements a number of times until the portion of vessel is cleared. The starting position for the exemplary process of this embodiment is shown in FIG. 6A with subsequent steps shown in subsequent FIGS where motions in directions 251-261 are taken in sequence. In this embodiment, axial movement occurs only in the distal direction along the length of the vessel. In this embodiment, for illustrative purposes the vessel is considered to be cleared after only left and right radial transitions and associated axial sweeps but in a vessel having a y-direction (i.e. in and out of the page) that is greater than the diameter of the ablation tool additional radial increments will occur if it is desired to further clear the vessel. In some alternative embodiments, both expansion devices 112 and 122 may be located on the same side (e.g. the proximal side) of the obstruction. In some alternative embodiments, instead distal and proximal axial motions leading to removal of material, the ablation tool start operations at one of the proximal or distal ends and then removal operations may be made to substantially occur in only a single direction (e.g. during proximal sweeps or during distal sweeps).

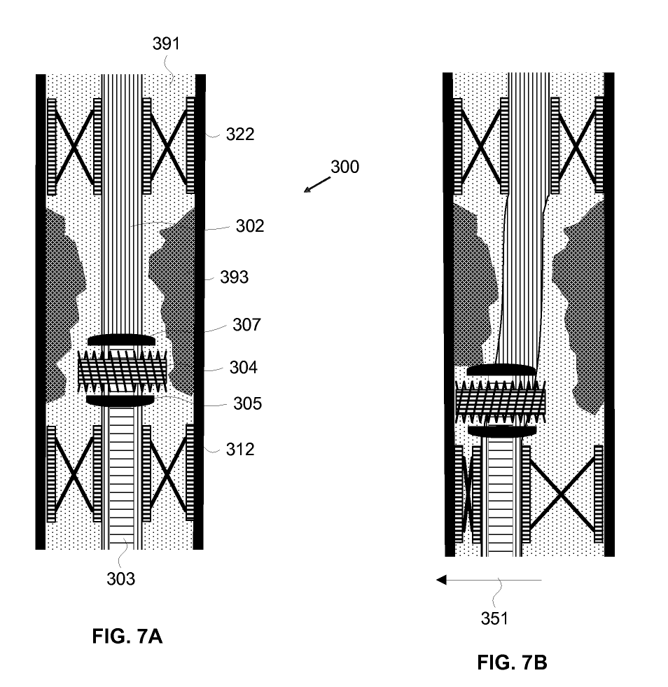

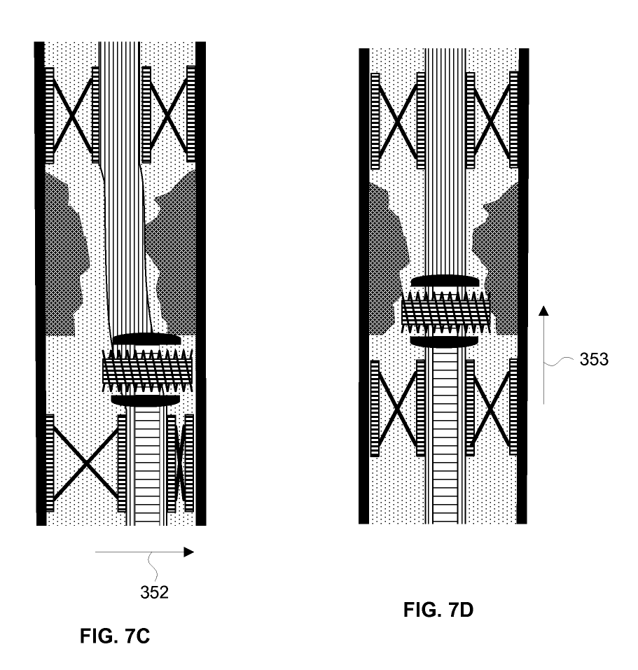

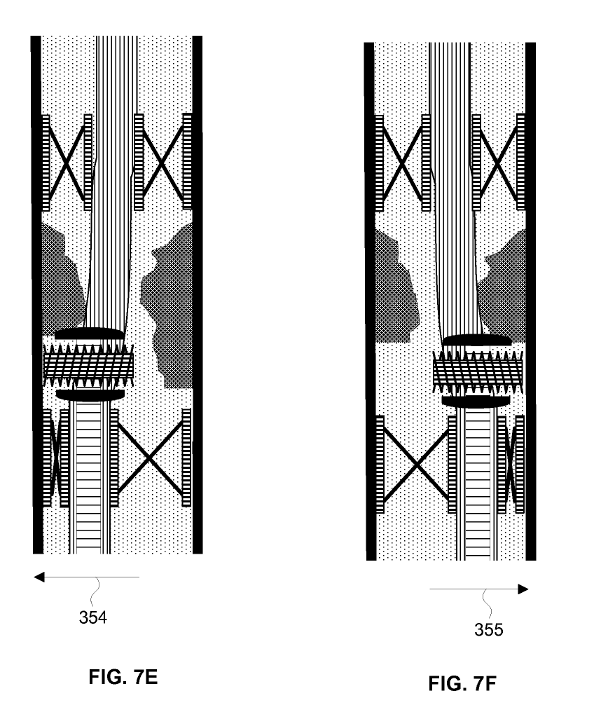

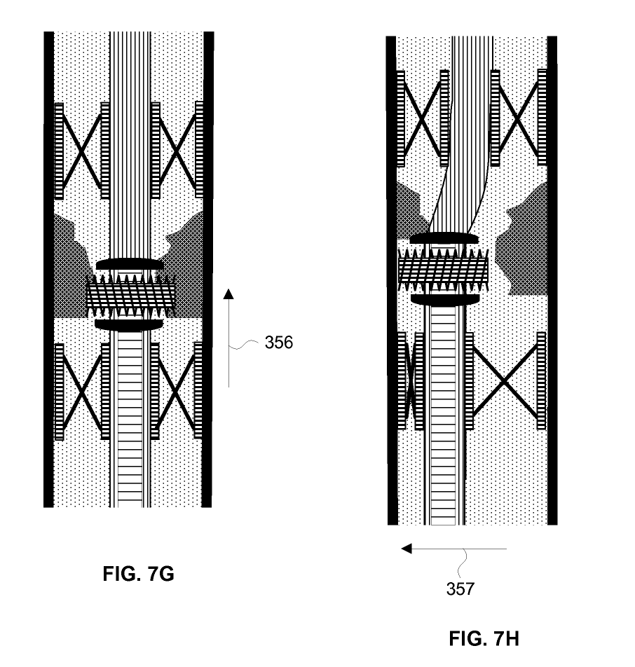

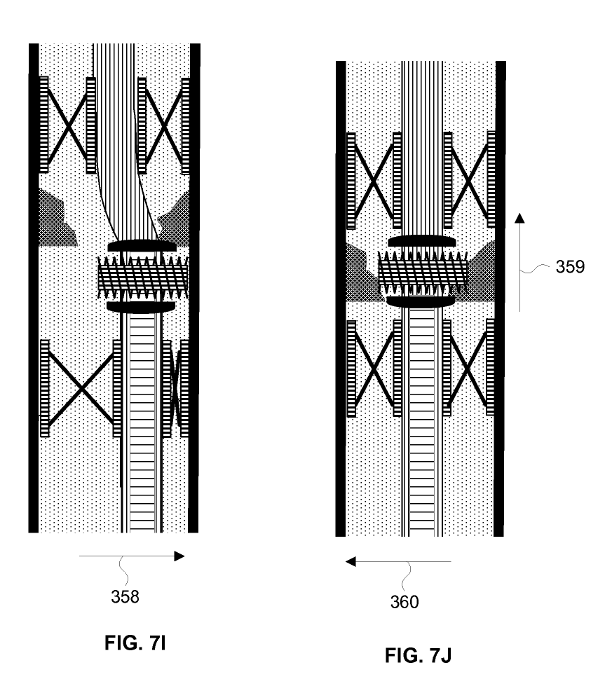

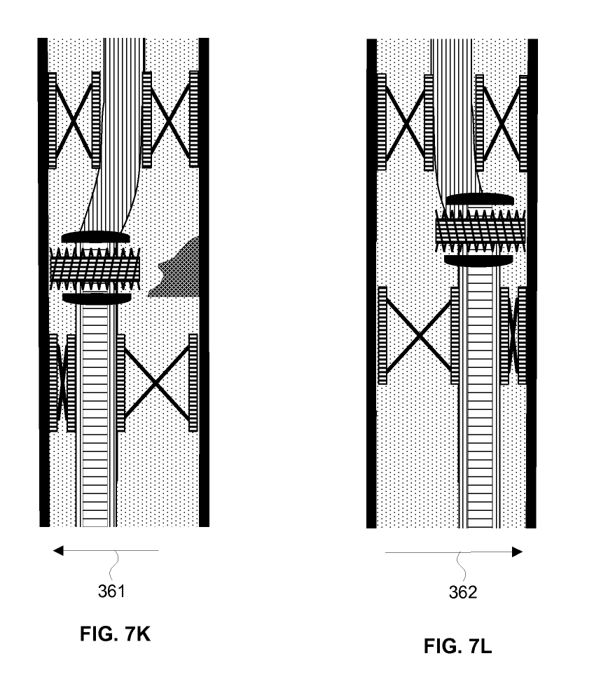

FIGS. 7A-7L provide schematic illustrations of a process for performing an atherectomy or thrombectomy using an atherectomy device or thrombectomy device 300 having an ablation device 304, with optional caps 305 and 307, and a first stabilization element 312 located on the proximal side of the an obstruction 393 in vessel 391 and a second stabilization device 322 located on the distal side of the obstruction and where the device is made to clean a radial area followed by a release of the anchoring of the proximal stabilization device, distal incrementing and re-anchoring of the proximal stabilization device in proximity to the remaining obstruction, and then a repeating of the radial and axial movements a number of times until the portion of vessel is cleared. The ablation device is made to move relative to the vessel via axial movements of the proximal stabilizer 312 relative to the distal stabilizer 322 and via radial movements associated with alternating expansions and contractions of the proximal stabilizers 312 while the radial position of the distal side of the catheter or guide wire 302 is held in a fixed position by the distal stabilization elements 322. Control element 303 may be used, alone or in conjunction with other elements, to control motion of the ablation device and may be used to control motion of the stabilizers. The starting position for the exemplary process of this embodiment is shown in FIG. 7A with subsequent steps shown in subsequent FIGS where motions in directions 351-361 are taken in sequence. In this embodiment, guide wires and/or linkages connect the proximal and distal stabilization devices and extend through the central portion of the ablation tool to aid in guidance of the ablation tool and to pass control signals to the distal stabilization element and to/or from any other distal elements. In this embodiment, axial movement occurs only in the distal direction along the length of the vessel. In this embodiment, for illustrative purposes the vessel is considered to be cleared after only left and right radial transitions and associated axial sweeps but in a vessel having a y-direction (i.e. in and out of the page) that is greater than the diameter of the ablation tool additional radial increments will occur if it is desired to further clear the vessel. In some alternative embodiments, the proximal and distal device may stay in fixed positions during the axial incrementing (where the ablation tool can be incremented axially along the linkage). In some alternative embodiments, instead to the distal stabilization devices having a fixed axial position during radial movements of the proximal stabilization device it may be made to undergo matching or partially matching radial incremental movements. In some alternative embodiments, both expansion devices may be located on the same side (e.g. the proximal side) of the obstruction with both stabilization elements held fixed through obstruction clearing, alternatively the closer stabilization element may be walked axially toward the distally receding obstruction or after each radial segment is cleared or after a given number of radial segments are cleared or a given axial length is cleared. Alternatively, both proximally positioned stabilization devices may undergo and alternating or simultaneous distal walking incremental motion.

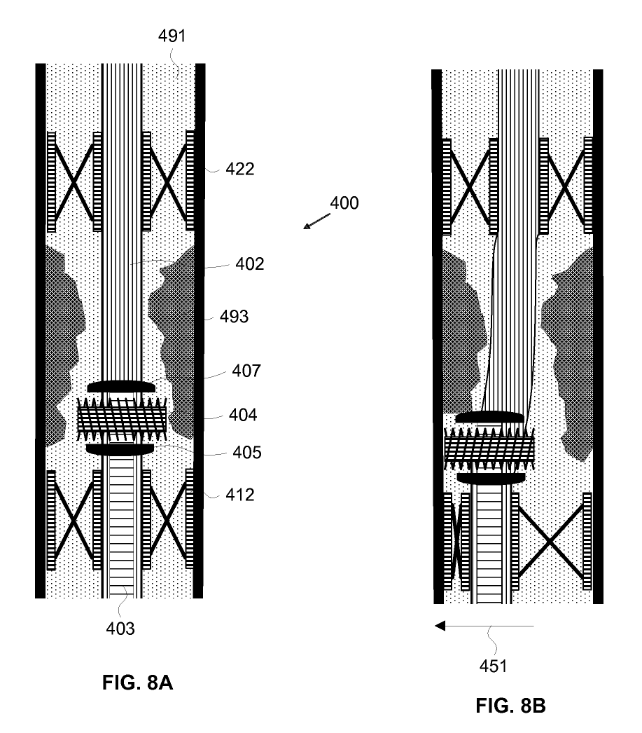

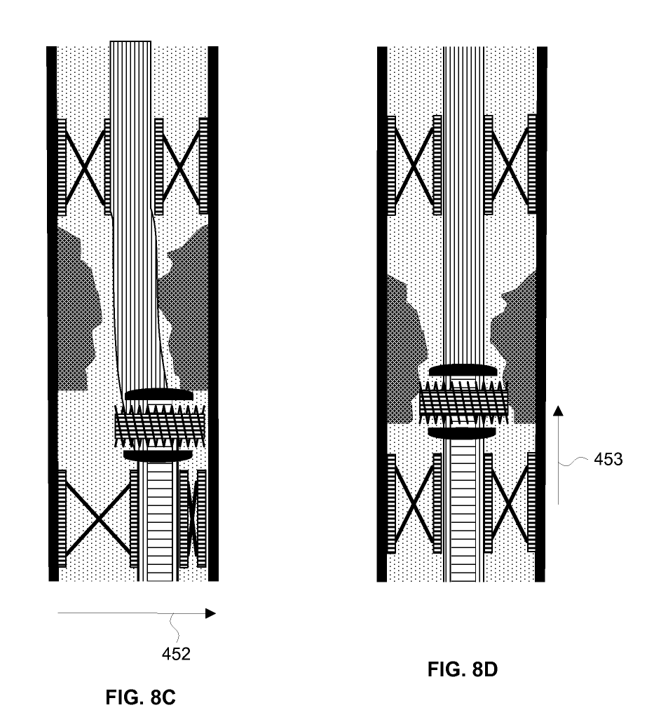

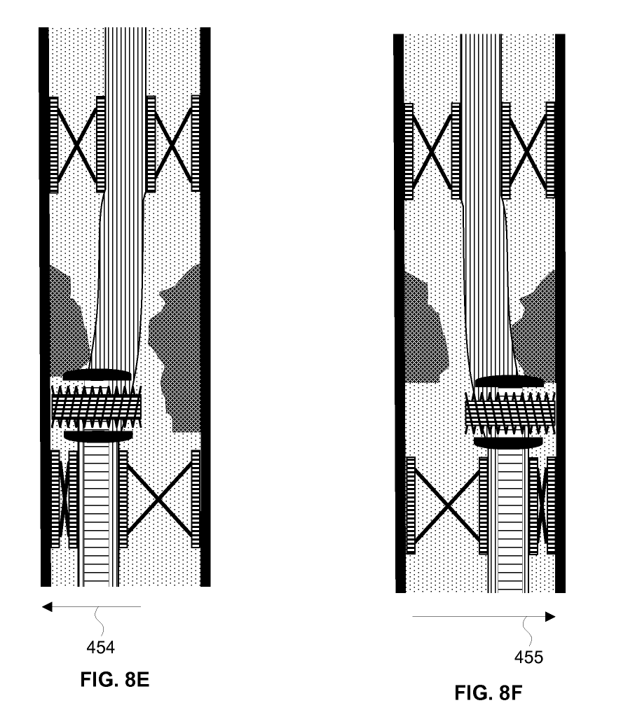

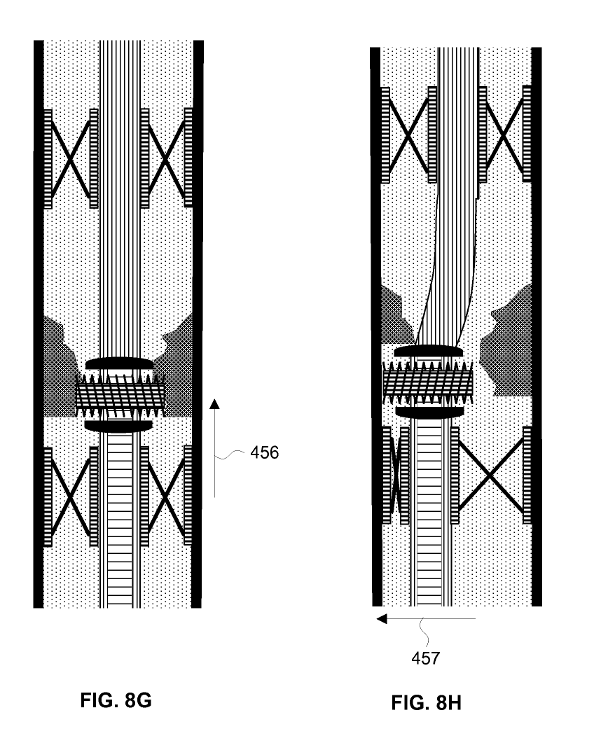

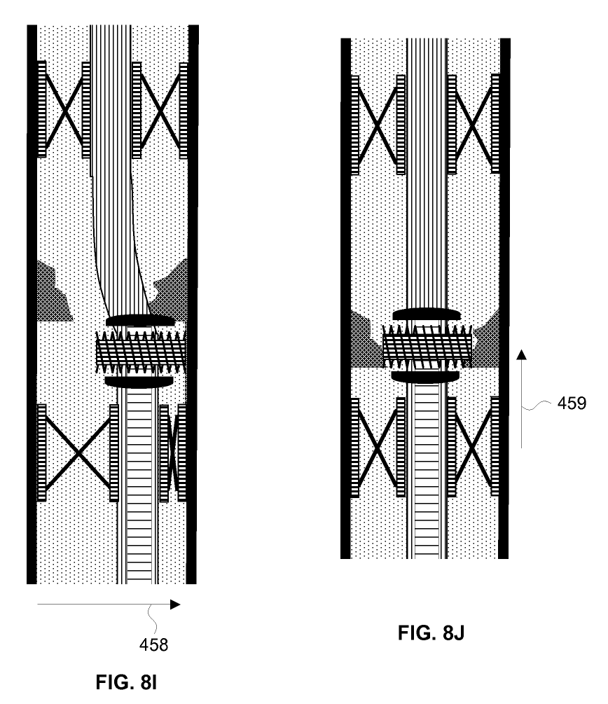

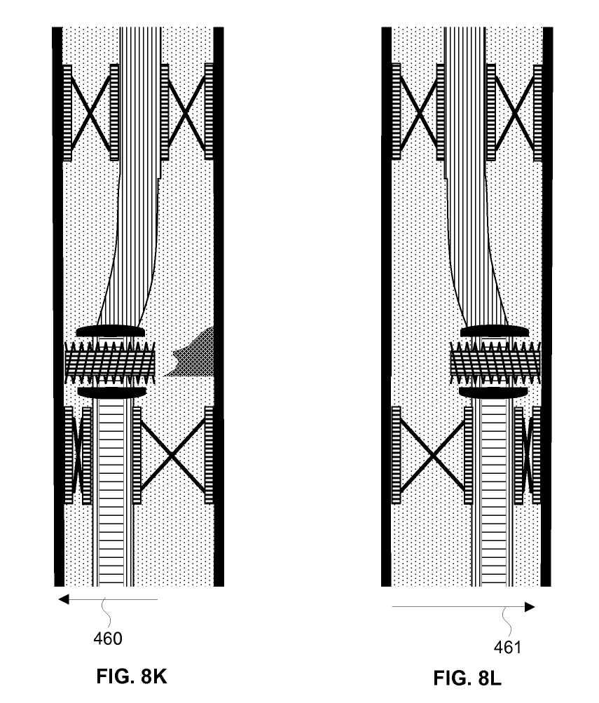

FIGS. 8A-8L illustrate a fourth use method of the invention for performing an atherectomy or a thrombectomy which is a variation of the embodiment of FIGS. 7A-7L. These FIGS. provide schematic illustrations of a fourth process for performing an atherectomy or thrombectomy using an atherectomy device or thrombectomy device 400 having an ablation device 404, with optional end caps 405 and 407, and a first stabilization element 412 located on the proximal side of the an obstruction 493 in vessel 491 and a second stabilization device 422 located on the distal side of the obstruction and where the device is made to clean a radial area followed the simultaneous or alternating release of both the distal and proximal stabilization devices, distal incrementing of the stabilization devices and the ablation tool, and re-anchoring of the stabilization devices (with the proximal stabilization device being anchored in proximity to the remaining obstruction), and then a repeating of the radial and axial movements a number of times until the portion of vessel is cleared. The starting position for the exemplary process of this embodiment is shown in FIG. 8A with subsequent steps shown in subsequent FIGS where motions in directions 451-461 are taken in sequence. In this embodiment, catheter and/or guide wires 402 and/or linkages connect the proximal and distal stabilization devices and extend through the central portion of the ablation tool to aid in guidance of the ablation tool and to pass control signals to the distal stabilization element and to/or from any other distal elements. Control signals may be carrier via control element 403 which may also be used to operate the ablation device 404. In this embodiment, axial movement occurs only in the distal direction along the length of the vessel. In this embodiment, for illustrative purposes the vessel is considered to be cleared after only left and right radial transitions and associated axial sweeps but in a vessel having a y-direction (i.e. in and out of the page) that is greater than the diameter of the ablation tool additional radial increments will occur if it is desired to further clear the vessel. In some alternative embodiments, instead to the distal stabilization devices having a fixed axial position during radial movements of the proximal stabilization device it may be made to undergo matching or partially matching radial incremental movements.

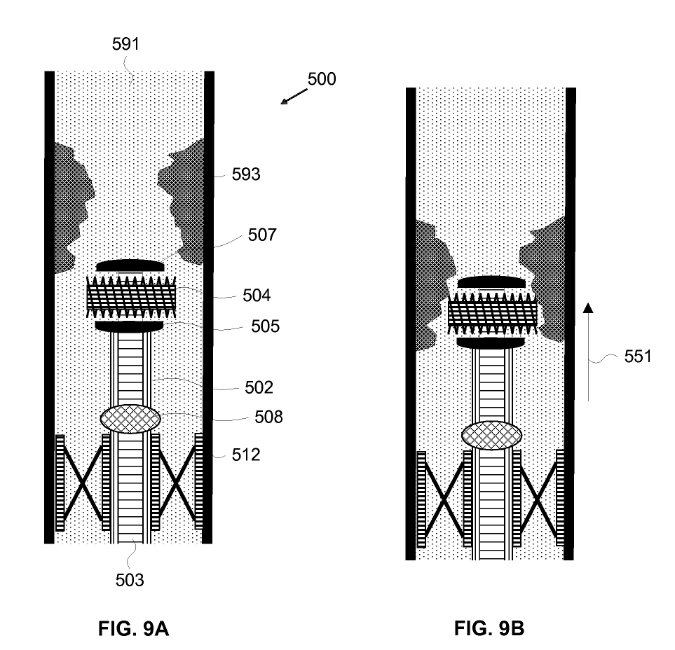

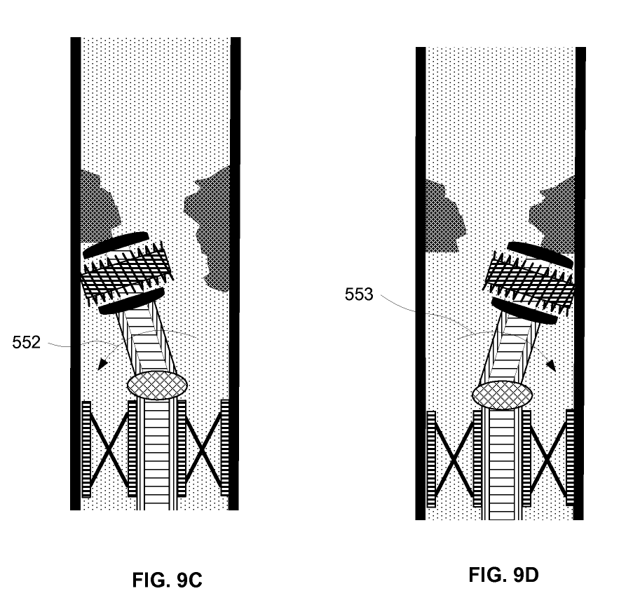

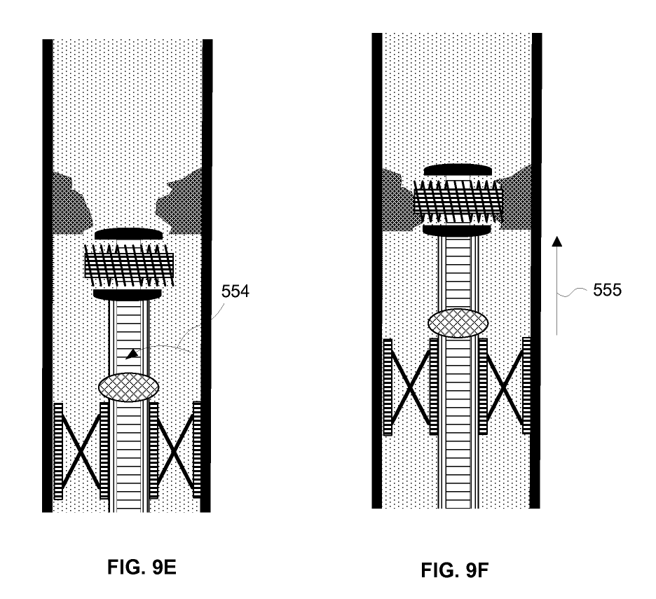

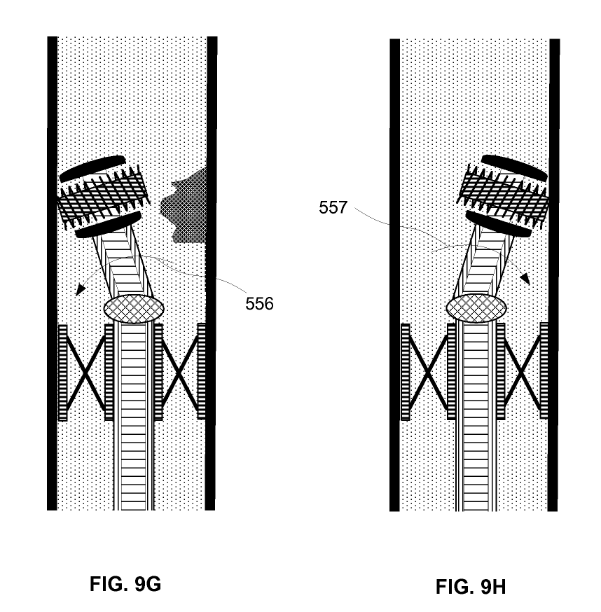

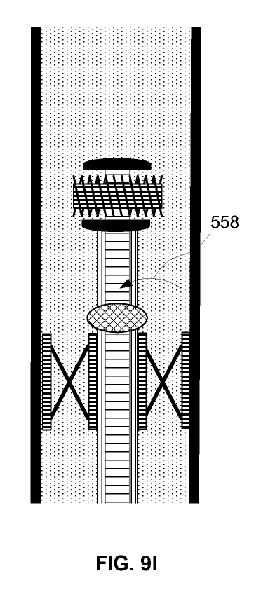

FIGS. 9A-9I provide schematic illustrations of a process of a fifth embodiment for performing an atherectomy or thrombectomy using a device 500 including an ablation tool 504 having proximal and distal caps 505 and 507 which is pivotal relative to a more proximal end of the catheter 502 via pivot 508 where movement of the tool may be made to occur via control element 503. The tool may be made to remove material 593 from the interior of vessel 593 via control pivoting of the tool 508 during tool rotation such that a desired cross-sectional portion of the vessel is swept clear after which the tool may be made to walk axially along the vessel by unseating, axially incrementing, and then reseating the proximal stabilizer 512 (followed by repeated rotation and pivoting. In the present embodiment, care may be taken either in the mechanical design of the device or in the control processes used such that the radial most etch of the ablation tool does not extend beyond a safer ablation or removal zone. In some variations of this embodiment, the tool may be made to have a shape (e.g. conical in the distal direction such that an extended axial swath is cleared when the device is tilted or pivoted at a desired angle.

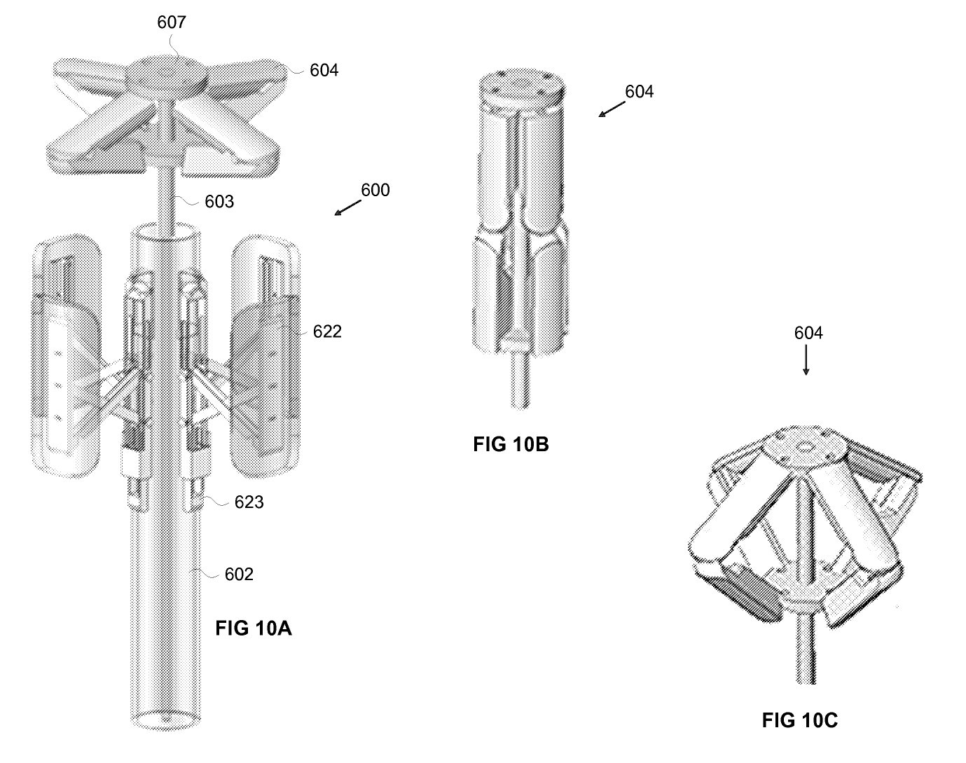

FIGS. 10A-10C provide perspective views of an atherectomy or thrombectomy device 600 according to a sixth embodiment of the invention where an expandable cutting or removal tool 604 is provided at the distal end of a catheter 602. The device also includes stabilization elements 622 with control elements 623. The removal tool 604 can be made to open by proximal tensioning the control element/wire 603 such that the proximal end of the tool contacts the distal end of catheter 602 or a distal stop (not shown) such that the proximal and distal ends of the tool are brought into proximity such that the arm extensions (i.e. cutting elements) are forced to more radial positions. Similarly, the pads of stabilizers 622 can be made to move radially in and out by axial actuation of control element 623 via wires, push elements, or other pull elements (not shown). The tool of this embodiment may be used in place of the ablation tools referred to in the first through fifth embodiments with appropriate modifications to allow control of distal elements (e.g. via through passages and the like. Rotation of tool 604 may be made to occur via rotation of control element 603, rotation of a sheath or other control element that may be made to engage the tool, via fluid flow against a turbine blades (not shown) or the like. In some variations of this embodiment, open and closed positions of the tool blades and the stabilizers may be set via spring loading or the like while the opposite position may be obtained via external actuation.

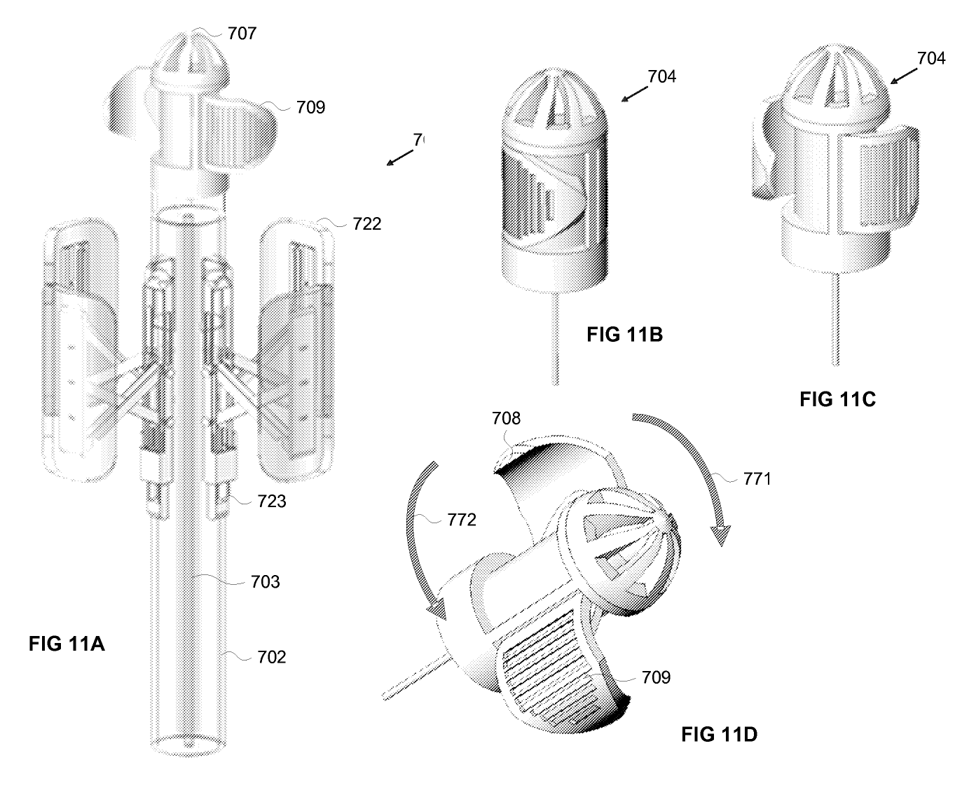

FIGS. 11A-11C provide perspective views of an atherectomy or thrombectomy device 700 according to a seventh embodiment of the invention which includes a cutting or abrading head 709 with an abrading tip 707 which is located at the distal end of a catheter 702 and can be made to open and close via a control element 703 (which may be used to drive wings or other expanding element from compact to extended radial positions). The device also includes stabilizers 622 which each have control elements 723. As can be seen in FIG. 11D rotation in one direction 771 can cause surfaces 709 to grind against blockage material to wear it away while rotation in direction 772 can cause tips 708 to encounter blockage material to scope it away. In some uses, rotation in direction 772 may be useful for removing a thrombus while rotation in direction 771 may be more useful for removing hardened atheroma. FIGS. 11B-11D depict the tool in various states of expansion. In some embodiments the orientation of the cutting blades may set to cause removed material to be force in a proximal or distal direction, e.g. toward a vacuum removal system or into a trap.

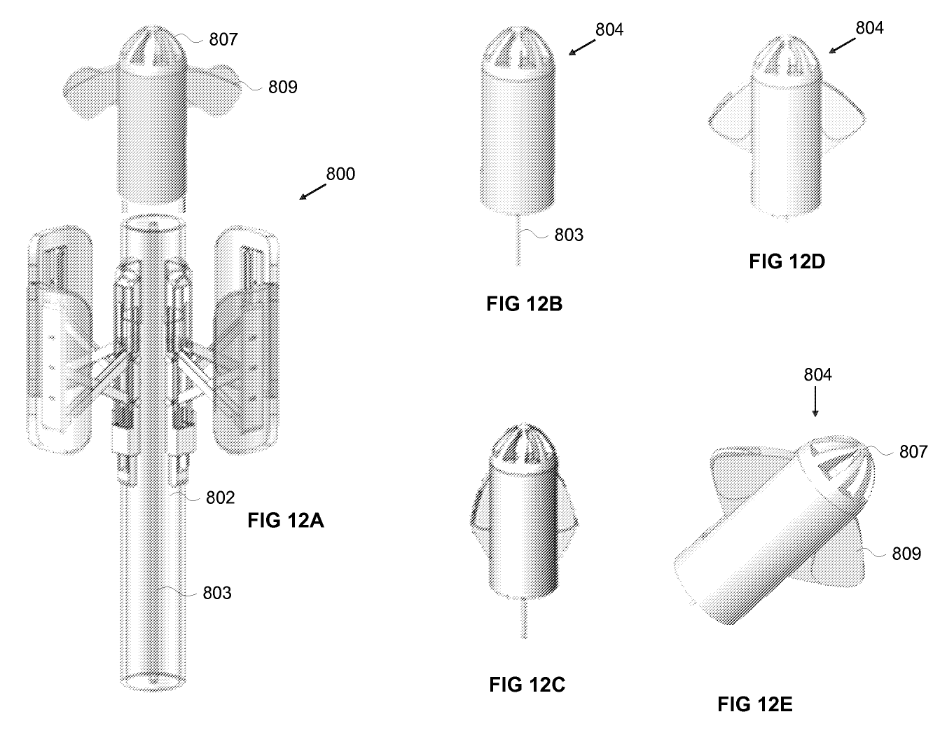

FIGS. 12A-12C provide perspective views of an atherectomy or thrombectomy device according to an eighth embodiment of the invention. The device of FIGS. 12A-12E has some similarities to that of the devices of the sixth and seventh embodiments. Similar elements are marked with similar numbers. The cutting head includes wings 809 and a cutting tip 807 wherein the wings can be made to open and closed based on actuation of control element 803.

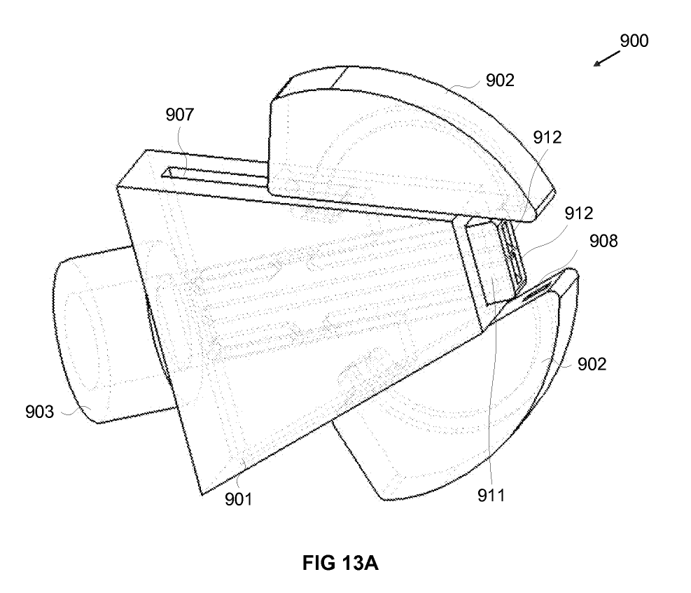

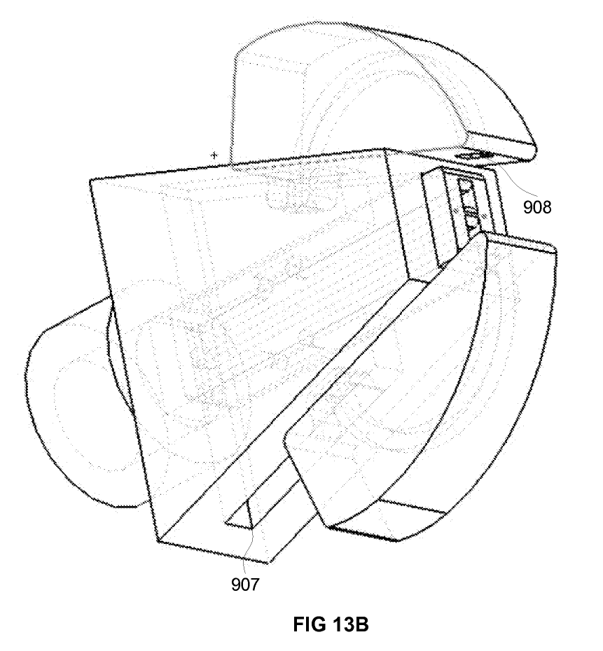

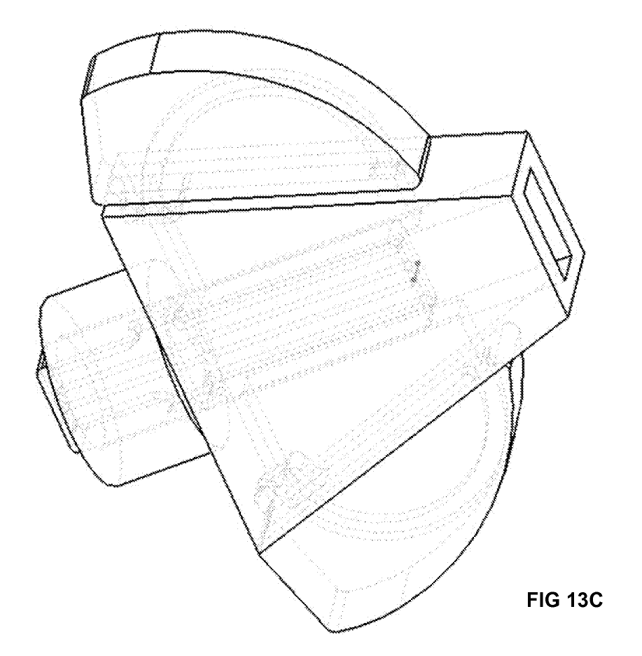

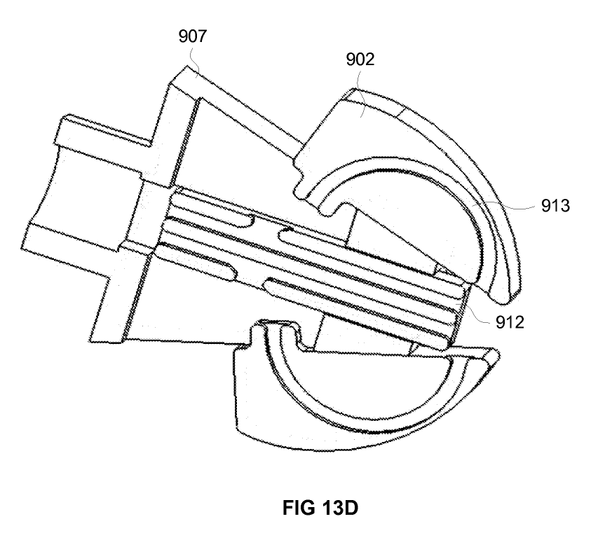

FIGS. 13A-13D provide perspective views of an atherectomy or thrombectomy device 900 according to a ninth embodiment of the invention wherein winged elements are not made by forcing axial elements into more proximal positions relative to one another which causes spreading of linked elements but instead via the sliding of wings 902 in a proximal direction relative to a central body 901 (which has an expanding configuration in the proximal direction). As illustrated each wing 902 engages central body via slot 907 through a wire (not shown) whose ends both extend out the proximal end of the device (i.e. beyond coupler 903) and which extends through slot 907 through path 913, out hole 908, and then through the path 912 in slide 911 (which can move proximally and distally relative to central body 901). In some embodiments, the wire elements may be made of NiTi or any other material that offers appropriate strength and flexibility. Slide 911 may be biased in one direction or the other via springs or the like or may be movable distally via a push tube or the like. During operation, the wings would be pulled back to their proximal position and the device rotated about its axis. Numerous variations of this embodiment are possible such as using linkages pull bars or wires in combination with passages in the wings that provide for proximal and spreading motion as the bars or wires are tensioned.

Further Comments and Conclusions

Structural or sacrificial dielectric materials may be incorporated into embodiments of the present invention in a variety of different ways. Such materials may form a third material or higher deposited on selected layers or may form one of the first two materials deposited on some layers. Additional teachings concerning the formation of structures on dielectric substrates and/or the formation of structures that incorporate dielectric materials into the formation process and possibility into the final structures as formed are set forth in a number of patent applications filed Dec. 31, 2003. The first of these filings is U.S. Patent Application No. 60/534,184 which is entitled “Electrochemical Fabrication Methods Incorporating Dielectric Materials and/or Using Dielectric Substrates”. The second of these filings is U.S. Patent Application No. 60/533,932, which is entitled “Electrochemical Fabrication Methods Using Dielectric Substrates”. The third of these filings is U.S. Patent Application No. 60/534,157, which is entitled “Electrochemical Fabrication Methods Incorporating Dielectric Materials”. The fourth of these filings is U.S. Patent Application No. 60/533,891, which is entitled “Methods for Electrochemically Fabricating Structures Incorporating Dielectric Sheets and/or Seed layers That Are Partially Removed Via Planarization”. A fifth such filing is U.S. Patent Application No. 60/533,895, which is entitled “Electrochemical Fabrication Method for Producing Multi-layer Three-Dimensional Structures on a Porous Dielectric”. Additional patent filings that provide teachings concerning incorporation of dielectrics into the EFAB process include U.S. patent application Ser. No. 11/139,262, filed May 26, 2005 by Lockard, et al., and which is entitled “Methods for Electrochemically Fabricating Structures Using Adhered Masks, Incorporating Dielectric Sheets, and/or Seed Layers that are Partially Removed Via Planarization”; and U.S. patent application Ser. No. 11/029,216, filed Jan. 3, 2005 by Cohen, et al., and which is entitled “Electrochemical Fabrication Methods Incorporating Dielectric Materials and/or Using Dielectric Substrates”. These patent filings are each hereby incorporated herein by reference as if set forth in full herein.

Some embodiments may employ diffusion bonding or the like to enhance adhesion between successive layers of material. Various teachings concerning the use of diffusion bonding in electrochemical fabrication processes are set forth in U.S. patent application Ser. No. 10/841,384 which was filed May 7, 2004 by Cohen et al. which is entitled “Method of Electrochemically Fabricating Multilayer Structures Having Improved Interlayer Adhesion” and which is hereby incorporated herein by reference as if set forth in full. This application is hereby incorporated herein by reference as if set forth in full.

Some embodiments may incorporate elements taught in conjunction with other medical devices as set forth in various U.S. patent applications filed by the owner of the present application and/or may benefit from combined use with these other medical devices: Some of these alternative devices have been described in the following previously filed patent applications: (1) U.S. patent application Ser. No. 11/478,934 (Docket No. P-US161-A-MF), filed Jun. 29, 2006, by Cohen et al., and entitled “Electrochemical Fabrication Processes Incorporating Non-Platable Materials and/or Metals that are Difficult to Plate On”; (2) U.S. Patent application Ser. No. 11/582,049 (Docket No. P-US164-A-MF), filed Oct. 16, 2006, by Cohen, and entitled “Discrete or Continuous Tissue Capture Device and Method for Making”; (3) U.S. patent application Ser. No. 11/625,807 (Docket No. P-US171-A-MF), filed Jan. 22, 2007, by Cohen, and entitled “Microdevices for Tissue Approximation and Retention, Methods for Using, and Methods for Making”; (4) U.S. patent application Ser. No. 11/696,722 (Docket No. P-US175-A-MF), filed Apr. 4, 2007, by Cohen, and entitled “Biopsy Devices, Methods for Using, and Methods for Making”; (5) U.S. patent application Ser. No. 11/734,273 (Docket No. P-US177-B-MF), filed Apr. 11, 2007, by Cohen, and entitled “Thrombectomy Devices and Methods for Making”; (6) U.S. Patent Application No. 60/942,200 (Docket No. P-US178-A-MF), filed Jun. 5, 2007, by Cohen, and entitled “Micro-Umbrella Devices for Use in Medical Applications and Methods for Making Such Devices”; (7) U.S. patent application Ser. No. 11/444,999 (Docket No. P-US159-A-MF), filed May 31, 2006, by Cohen, and entitled “Microtools and Methods for Fabricating Such Tools”; (8) U.S. patent application Ser. No. 11/734,256 (Docket No. P-US177-A-MF), by Cohen, filed Apr. 11, 2007, and entitled “Thrombectomy Devices and Methods for Making”; (9) U.S. patent application Ser. No. 11/734,273 (Docket No. P-US-177-B-MF), by Cohen, filed Apr. 11, 2007, and entitled “Thrombectomy Devices and Methods for Making”; (10) U.S. Patent Application No. 60/943,310 (Docket No. P-US180-A-MF), by Wu, filed Jun. 12, 2007, and entitled “Micro-scale and Meso-scale Expansion Tools for Medical Applications and Methods for Making”; (11) U.S. Patent Application No. 60/949,850 (Docket No. P-US180-B-MF), by Wu, filed Jul. 14, 2007, and entitled “Micro-scale and Meso-scale Expansion Tools for Medical Applications and Methods for Making”; (12) U.S. Patent Application No. 60/951,711 (Docket No. P-US180-C-MF), by Wu, filed Jul. 24, 2007, and entitled “Micro-scale and Meso-scale Expansion Tools for Medical Applications and Methods for Making”; (13) U.S. Patent Application No. 60/968,042 (Docket No. P-US180-D-MF), by Wu, filed Aug. 24, 2007, and entitled “Micro-scale and Meso-scale Expansion Tools for Medical Applications and Methods for Making”; (14) U.S. Patent Application No. 60/943,817 (Docket No. P-US182-A-MF), by Cohen, filed Jun. 13, 2007, and entitled “Micro-scale and Meso-scale Hydraulic and Pneumatic Tools for Medical Applications and Methods for Making”; (15) U.S. Patent Application No. 60/968,863 (Docket No. P-US182-B-MF), by Cohen, filed Aug. 29, 2007, and entitled “Micro-scale and Meso-scale Hydraulic and Pneumatic Tools for Medical Applications and Methods for Making”; (16) U.S. Patent Application No. 60/943,314 (Docket No. P-US183-A-MF), by Cohen, filed Jun. 12, 2007, and entitled “Miscellaneous Tools and Methods for Medical Applications”; (17) U.S. Patent Application No. 60/944,461 (Docket No. P-US184-A-MF), by Wu, filed Jun. 15, 2007, and entitled “Micro-scale and Meso-Scale Devices and Tools for Medical Applications and Methods for Making”; (18) U.S. Patent Application No. 60/948,262 (Docket No. P-US186-A-MF), by Frodis, filed Jul. 6, 2007, and entitled “Micro-Scale and Meso-Scale Hydraulically or Pneumatically Powered Devices Capable of Rotational”; (19) U.S. Patent Application No. 60/951,707 (Docket No. P-US187-A-MF), by Cohen, filed Jul. 24, 2007, and entitled “Advanced Guidewires”; and (20) U.S. Patent Application No. 60/968,043 (Docket No. P-US189-A-MF), by Cohen, filed Aug. 24, 2007, and entitled “Reconfigurable Articulating Wires”. Each of these applications is incorporated herein by reference as if set forth in full herein.

Though the embodiments explicitly set forth herein have considered multi-material layers to be formed one after another. In some embodiments, it is possible to form structures on a layer-by-layer basis but to deviate from a strict planar layer on planar layer build up process in favor of a process that interlaces material between the layers. Such alternative build processes are disclosed in U.S. application Ser. No. 10/434,519, filed on May 7, 2003, entitled Methods of and Apparatus for Electrochemically Fabricating Structures Via Interlaced Layers or Via Selective Etching and Filling of Voids. The techniques disclosed in this referenced application may be combined with the techniques and alternatives set forth explicitly herein to derive additional alternative embodiments. In particular, the structural features are still defined on a planar-layer-by-planar-layer basis but material associated with some layers are formed along with material for other layers such that interlacing of deposited material occurs. Such interlacing may lead to reduced structural distortion during formation or improved interlayer adhesion. This patent application is herein incorporated by reference as if set forth in full.

The patent applications and patents set forth below are hereby incorporated by reference herein as if set forth in full. The teachings in these incorporated applications can be combined with the teachings of the instant application in many ways: For example, enhanced methods of producing structures may be derived from some combinations of teachings, enhanced structures may be obtainable, enhanced apparatus may be derived, and the like.

| US Pat App No., Filing Date | |
|---|---|
| US App Pub No., Pub Date | |
| US Patent No., Pub Date | Inventor, Title |
| 09/493,496 - Jan. 28, 2000 | Cohen, “Method For Electrochemical Fabrication” |
| U.S. Pat. No. 6,790,377 - Sep. 14, 2004 | |
| 10/677,556 - Oct. 1, 2003 | Cohen, “Monolithic Structures Including Alignment and/or |
| 2004-0134772 - Jul. 15, 2004 | Retention Fixtures for Accepting Components” |
| 10/830,262 - Apr. 21, 2004 | Cohen, “Methods of Reducing Interlayer Discontinuities in |
| 2004-0251142A - Dec. 16, 2004 | Electrochemically Fabricated Three-Dimensional Structures” |
| U.S. Pat. No. 7,198,704 - Apr. 3, 2007 | |
| 10/271,574 - Oct. 15, 2002 | Cohen, “Methods of and Apparatus for Making High Aspect |
| 2003-0127336A - Jul. 10, 2003 | Ratio Microelectromechanical Structures” |
| U.S. Pat. No. 7,288,178 - Oct. 30, 2007 | |
| 10/697,597 - Dec. 20, 2002 | Lockard, “EFAB Methods and Apparatus Including Spray |
| 2004-0146650A - Jul. 29, 2004 | Metal or Powder Coating Processes” |
| 10/677,498 - Oct. 1, 2003 | Cohen, “Multi-cell Masks and Methods and Apparatus for |
| 2004-0134788 - Jul. 15, 2004 | Using Such Masks To Form Three-Dimensional Structures” |
| U.S. Pat. No. 7,235,166 - Jun. 26, 2007 | |
| 10/724,513 - Nov. 26, 2003 | Cohen, “Non-Conformable Masks and Methods and |
| 2004-0147124 - Jul. 29, 2004 | Apparatus for Forming Three-Dimensional Structures” |
| U.S. Pat. No. 7,368,044 - May 6, 2008 | |
| 10/607,931 - Jun. 27, 2003 | Brown, “Miniature RF and Microwave Components and |
| 2004-0140862 - Jul. 22, 2004 | Methods for Fabricating Such Components” |
| U.S. Pat. No. 7,239,219 - Jul. 3, 2007 | |
| 10/841,100 - May 7, 2004 | Cohen, “Electrochemical Fabrication Methods Including Use |
| 2005-0032362 - Feb. 10, 2005 | of Surface Treatments to Reduce Overplating and/or |
| U.S. Pat. No. 7,109,118 - Sep. 19, 2006 | Planarization During Formation of Multi-layer Three- |
| | Dimensional Structures” |
| 10/387,958 - Mar. 13, 2003 | Cohen, “Electrochemical Fabrication Method and |
| 2003-022168A - Dec. 4, 2003 | Application for Producing Three-Dimensional Structures |
| | Having Improved Surface Finish“ |
| 10/434,494 - May 7, 2003 | Zhang, “Methods and Apparatus for Monitoring Deposition |
| 2004-0000489A - Jan. 1, 2004 | Quality During Conformable Contact Mask Plating |
| | Operations” |
| 10/434,289 - May 7, 2003 | Zhang, “Conformable Contact Masking Methods and |
| 20040065555A - Apr. 8, 2004 | Apparatus Utilizing In Situ Cathodic Activation of a |
| | Substrate” |
| 10/434,294 - May 7, 2003 | Zhang, “Electrochemical Fabrication Methods With |
| 2004-0065550A - Apr. 8, 2004 | Enhanced Post Deposition Processing” |
| 10/434,295 - May 7, 2003 | Cohen, “Method of and Apparatus for Forming Three- |
| 2004-0004001A - Jan. 8, 2004 | Dimensional Structures Integral With Semiconductor Based |
| | Circuitry” |
| 10/434,315 - May 7, 2003 | Bang, “Methods of and Apparatus for Molding Structures |
| 2003-0234179 A - Dec. 25, 2003 | Using Sacrificial Metal Patterns” |
| U.S. Pat. No. 7,229,542 - Jun. 12, 2007 | |
| 10/434,103 - May 7, 2004 | Cohen, “Electrochemically Fabricated Hermetically Sealed |
| 2004-0020782A - Feb. 5, 2004 | Microstructures and Methods of and Apparatus for |
| U.S. Pat. No. 7,160,429 - Jan. 9, 2007 | Producing Such Structures” |
| 10/841,006 - May 7, 2004 | Thompson, “Electrochemically Fabricated Structures Having |
| 2005-0067292 - May 31, 2005 | Dielectric or Active Bases and Methods of and Apparatus for |
| | Producing Such Structures” |
| 10/434,519 - May 7, 2003 | Smalley, “Methods of and Apparatus for Electrochemically |
| 2004-0007470A - Jan. 15, 2004 | Fabricating Structures Via Interlaced Layers or Via Selective |
| U.S. Pat. No. 7,252,861 - Aug. 7, 2007 | Etching and Filling of Voids” |
| 10/724,515 - Nov. 26, 2003 | Cohen, “Method for Electrochemically Forming Structures |
| 2004-0182716 - Sep. 23, 2004 | Including Non-Parallel Mating of Contact Masks and |
| U.S. Pat. No. 7,291,254 - Nov. 6, 2007 | Substrates” |
| 10/841,347 - May 7, 2004 | Cohen, “Multi-step Release Method for Electrochemically |
| 2005-0072681 - Apr. 7, 2005 | Fabricated Structures” |
| 60/533,947 - Dec. 31, 2003 | Kumar, “Probe Arrays and Method for Making” |
| 60/534,183 - Dec. 31, 2003 | Cohen, “Method and Apparatus for Maintaining Parallelism |
| | of Layers and/or Achieving Desired Thicknesses of Layers |
| | During the Electrochemical Fabrication of Structures” |
| 11/733,195 - Apr. 9, 2007 | Kumar, “Methods of Forming Three-Dimensional Structures |
| 2008-0050524 - Feb. 28, 2008 | Having Reduced Stress and/or Curvature” |
| 11/506,586 - Aug. 8, 2006 | Cohen, “Mesoscale and Microscale Device Fabrication |
| 2007-0039828 - Feb. 22, 2007 | Methods Using Split Structures and Alignment Elements” |
| 10/949,744 - Sep. 24, 2004 | Lockard, “Three-Dimensional Structures Having Feature |
| 2005-0126916 - Jun. 16,2005 | Sizes Smaller Than a Minimum Feature Size and Methods |
| U.S. Pat. No. 7,498,714 - Mar. 3, 2009 | for Fabricating” |

Though various portions of this specification have been provided with headers, it is not intended that the headers be used to limit the application of teachings found in one portion of the specification from applying to other portions of the specification. For example, it should be understood that alternatives acknowledged in association with one embodiment, are intended to apply to all embodiments to the extent that the features of the different embodiments make such application functional and do not otherwise contradict or remove all benefits of the adopted embodiment. Various other embodiments of the present invention exist. Some of these embodiments may be based on a combination of the teachings herein with various teachings incorporated herein by reference.

In view of the teachings herein, many further embodiments, alternatives in design and uses of the embodiments of the instant invention will be apparent to those of skill in the art. As such, it is not intended that the invention be limited to the particular illustrative embodiments, alternatives, and uses described above but instead that it be solely limited by the claims presented hereafter.

We claim:

1. A procedure for removing material from interior walls of a vessel without damaging the walls of the vessel in the region from which material is to be removed, comprising:
(a) supplying a catheter;
(b) supplying an removal tool and at least one radial stabilizer, wherein the radial stabilizer comprises a central body and a plurality of extendable elements that can contact the walls, wherein the removal tool is located beyond the distal end of the catheter, or can be made to extend from the distal end of the catheter, and wherein the removal tool and stabilizer are in a fixed or controllable position relative to one another;
(c) inserting the catheter into the vessel of the patient such that the removal tool and stabilizer are located in proximity to a region of material to be removed;
(d) expanding the at least one stabilizer to fix the radial position of the removal tool relative to the vessel walls;
(e) activating the removal tool;
(f) adjusting the radial position of the removal tool relative to the vessel walls via movement of the central body of the stabilizer relative to the walls of the vessel to bring the removal tool in contact with the material to be removed and to remove at least a portion of the material;
(g) adjusting the radial position of the removal tool via movement of the stabilizer while the stabilizer is anchored so as to remove material and adjusting the axial position of the removal tool with or without the stabilizer being anchored so as to position the removal tool to remove further material; and
(h) repeating the radial and axial movements of the removal tool to remove a desired quantity of material from the vessel.

2. The procedure of claim 1 wherein the removal tool includes a head with removal elements that can be made to extend and contract in the radial direction.

3. The procedure of claim 2 wherein the expansion occurs via sliding the extension elements against a sloped surface.

4. The procedure of claim 2 wherein the expansion occurs via bring separated portions of the head into more proximate positions relative to one another such that other portions are forced into more radially extended positions.

5. A procedure for removing material from interior walls of a vessel without damaging the walls of the vessel in the region from which material is to be removed, comprising:
(a) supplying a catheter;
(b) supplying an removal tool and at least one radial stabilizer, wherein the radial stabilizer comprises a central body and a plurality of extendable elements that can contact the walls, wherein the removal tool is located beyond the distal end of the catheter, or can be made to extend from the distal end of the catheter, and wherein the removal tool and stabilizer are in a fixed or controllable position relative to one another;
(c) inserting the catheter into the vessel of the patient such that the removal tool and stabilizer are located in proximity to a region of material to be removed;
(d) expanding the at least one stabilizer to fix the radial position of the removal tool relative to the vessel walls;
(e) activating the removal tool;
(f) adjusting the radial position of the removal tool relative to the vessel walls via pivoting a head of the tool relative to another portion of the tool such that a radial sweeping of the tool can occur so as to bring the removal tool in contact with the material to be removed and to remove at least a portion of the material;
(g) adjusting the axial position of the removal tool; and
(h) repeating the radial and axial movements of the removal tool to remove a desired quantity of material from the vessel.
